(19) United States
(12) Patent Application Publication
(10) Pub. No.: US 2008/0172006 A1
(43) Pub. Date: Jul. 17, 2008

(54) Patency Check Compatible Check Valve And Fluid Delivery System Including The Patency Check Compatible Check Valve

(75) Inventor: Jeffrey H. Hicks, Lilburn, GA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 12/014,288

(22) Filed: Jan. 15, 2008

Related U.S. Application Data
(60) Provisional application No. 60/884,918, filed on Jan. 15, 2007.

(51) Int. Cl. A61M 5/31

(52) U.S. Cl. 604/249

(57) ABSTRACT

The check valve includes a housing body and a valve member. An actuator is associated with the valve member or housing body. The housing body defines a flow passage and an inlet port and an outlet port each communicating with the flow passage. The housing body includes a seal seat in the flow passage between the inlet port and outlet port. The valve member is disposed in the flow passage and is adapted to engage the seal seat. The actuator may be operatively connected to the valve member to place the valve member in an override position permitting bi-directional fluid flow through the flow passage. The valve member may be a cantilever valve member responsive to fluid flow in the flow passage. The valve member may have multiple states or positions. The actuator may be a bypass actuator selectively placing the inlet port in fluid communication with the outlet port.

### CROSS REFERENCE TO RELATED APPLICATIONS

This application claims an invention which was disclosed in Provisional Application No. 60/884,918, filed Jan. 15, 2007, entitled Patency Check Compatible Check Valve And Fluid Delivery System Including The Patency Check Compatible Check Valve. The provisional application is hereby claimed, and the aforementioned application is hereby incorporated herein by reference.

### RELATED APPLICATIONS

This Application may contain subject matter that is related to that disclosed or claimed in one or more of the two following U.S. Applications: application Ser. No. 10/722,370, filed Nov. 25, 2003 now U.S. publication No. US 2005-0113754, published May 25, 2005; and application Ser. No. 10/159,592 filed May 30, 2002, now U.S. publication No. US 2004-0064041, published Apr. 1, 2004; which may contain subject matter that is related to that disclosed or claimed in one or more of the following U.S. patents or applications: U.S. Pat. No. 6,652,489, filed on Feb. 5, 2001; application Ser. No. 10/159,592, filed on May 30, 2002; now U.S. publication No. US 2004-0064041, published Apr. 1, 2004; application Ser. No. 09/448,835, filed on Nov. 24, 1999; application Ser. No. 10/174,631, filed on Jun. 19, 2002, now U.S. Pat. No. 7,029,459 issued Apr. 18, 2006; application Ser. No. 10/619,137, filed on Jul. 14, 2003, now U.S. publication No. US 2004-0068223, published Apr. 8, 2004; application Ser. No. 10/668,643, filed on Sep. 23, 2003, now U.S. Publication No. US 2004-0133161, published Jul. 8, 2004; application Ser. No. 10/668,673, filed on Sep. 23, 2003, now U.S. Publication No. US 2004-0133162, published Jul. 8, 2004; application Ser. No. 10/669,144, filed on Sep. 23, 2003, now U.S. Publication No. US 2004-0116861, published Jun. 17, 2004; application Ser. No. 10/669,148, filed on Sep. 23, 2003, now U.S. Publication No. US 2004-0133153, published Jul. 8, 2004; application Ser. No. 10/670,154, filed on Sep. 23, 2003, now U.S. Publication No. US 2004-0133183, published Jul. 8, 2004; application Ser. No. 10/380,188, filed on Mar. 10, 2003, now U.S. Publication No. US 2004-0158205, published Aug. 12, 2004; Application Serial No. 09/765,498, filed on Jan. 18, 2001 now U.S. Pat. No. 7,018,363, issued Mar. 28, 2006; and application Ser. No. 10/606,157, filed on Jun. 25, 2003.

### BACKGROUND OF THE INVENTION

1. Field of the Invention

This invention is generally directed to the delivery of fluids in medical procedures and, more particularly, to valves used for fluid control actions in fluid delivery devices, systems, and methods used in medical procedures.

2. Description of Related Art

In many medical diagnostic and therapeutic procedures, a medical practitioner such as a physician injects a patient with a fluid. In recent years, a number of injector-actuated syringes and powered injectors for pressurized injection of fluids, such as contrast media (often referred to simply as “contrast”), have been developed for use in procedures such as angiography, computed tomography, ultrasound, and NMR/MRI. In general, these powered injectors are designed to deliver a preset amount of contrast at a preset flow rate.

Angiography is used in the detection and treatment of abnormalities or restrictions in blood vessels. In an angiographic procedure, a radiographic image of a vascular structure is obtained through the use of a radiographic contrast which is injected through a catheter. The vascular structures in fluid connection with the vein or artery in which the contrast is injected are filled with contrast. X-rays passing through the region of interest are absorbed by the contrast, causing a radiographic outline or image of blood vessels containing the contrast. The resulting images can be displayed on, for example, a video monitor and recorded.

In a typical angiographic procedure, the medical practitioner places a cardiac catheter into a vein or artery. The catheter is connected to either a manual or to an automatic contrast injection mechanism. A typical manual contrast injection mechanism includes a syringe in fluid connection with a catheter connection. The fluid path also includes, for example, a source of contrast, a source of flushing fluid, typically saline, and a pressure transducer to measure patient blood pressure. In a typical system, the source of contrast is connected to the fluid path via a valve, for example, a three-way stopcock. The source of saline and the pressure transducer may also be connected to the fluid path via additional valves, again such as stopcocks. The operator of the manual system controls the syringe and each of the valves to draw saline or contrast into the syringe and to inject the contrast or saline into the patient through the catheter connection.

Automatic contrast injection mechanisms typically include a syringe connected to a powered injector having, for example, a powered linear actuator. Typically, an operator enters settings into an electronic control system of the powered injector for a fixed volume of contrast and a fixed rate of injection. In many systems, there is no interactive control between the operator and the powered injector, except to start or stop the injection. A change in flow rate in such systems occurs by stopping the machine and resetting the injection parameters. Automation of angiographic procedures using powered injectors is discussed, for example, in U.S. Pat. Nos. 5,460,609; 5,573,515; and 5,800,397.

In fluid delivery procedures such as angiography, controlling the direction of fluid flow in the fluid path is important to ensure that the appropriate amount of contrast and saline, as examples, are delivered to the patient. In delivering such fluids to a patient through the fluid path, it is often important to ensure that the fluid moves in only one direction, generally from the fluid source to the patient. In order to prevent reverse fluid flow, check valves are incorporated into the fluid path at strategic locations to prevent such reverse fluid flow. Check valves are well-known structures that limit flow to one direction through a fluid line and include structure that allows fluid flow in one direction, while preventing fluid flow in the opposing direction. Some check valves used in the medical area in particular include and override mechanism associated with the internal structure to allow reverse fluid flow for certain purposes such as patency checks. However, such check valves are not the norm in fluid paths associated with fluid injection devices as it usually of higher importance to prevent reverse fluid flow in the fluid path for patient protection purposes.

During normal operation of a fluid delivery system, fluid flow is provided under pressure by a syringe injector to the fluid path which may include apparatus such as valves and like fluid control structures for managing the fluid flow through the fluid path to a catheter inserted into the patient. Prior to actually delivering fluid to the patient, it is often necessary during the set-up preparations for a fluid injection procedure to confirm the correct positioning of the catheter in a blood vessel or other body lumen. This is often determined by conducting a patency check with the fluid delivery apparatus. A patency check is conducted by actuating the syringe injector so that the syringe plunger is momentarily retracted until blood or another body fluid is detected in the tubing of the fluid path, thereby confirming correct catheter placement in a blood vessel. Typical check valves prevent this procedure from being conducted due to their one-directional flow path. Thus, in order to conduct a patency check, it desirable to temporarily override the check function of a check valve to allow reverse fluid in a fluid path. Typically, such an override function is operator-actuated to allow the reverse fluid flow from the output side of the check valve to the input side.

Numerous check valve examples are known in the art which are particularly adapted for use in medical fluid injection procedures. One such example is found in U.S. Pat. No. 6,988,510 to Enerson which discloses a free floating disk check valve which is quickly responsive to a closed position when backflow is experienced in a fluid line. U.S. Pat. Nos. 5,743,872 and 5,665,074 both to Kelly disclose a limited backflow reflux valve for use with a fluid injection system including a syringe, catheter, and bulk container of injection fluid. The reflux valve permits injection of fluid from the syringe through the catheter into the patient, and also permits refilling of the syringe from the bulk container without disconnection of any tubing. U.S. Patent Application Publication No. 2005/0194047 to Bausmith discloses a check valve arrangement for a dual-syringe fluid injection system. U.S. Pat. No. 6,390,130 to Guala discloses a disc check valve for a medical infusion line. U.S. Pat. No. 6,089,272 to Brand et al. and U.S. Pat. No. 5,992,462 to Atkinson disclose additional examples of disc check valve suitable for medical infusion lines. U.S. Pat. No. 5,727,594 to Choksi discloses several medical purpose check valves and a non-medical check valve embodiment which is the form of a free-floating type check. U.S. Pat. No. 5,593,385 to Harrison et al. discloses a ball check valve specifically suited for use with contrast media due its higher viscosity attributes. U.S. Pat. No. 5,692,539 to Pickl, Jr. discloses a spring-biased check valve for medical fluid delivery applications. U.S. Pat. No. 5,575,767 to Stevens discloses a spring-biased ball check valve specifically adapted for high fluid pressure angiography environments. U.S. Pat. No. 4,712,583 to Pelmulder et al.; U.S. Pat. No. 4,683,916 to Raines; and U.S. Pat. No. 4,415,003 to Paradis disclose additional disc check valves used in medical fluid delivery applications

As the Bausmith Publication indicates, fluid delivery platforms may include the use of multiple syringes. The use of multiple syringes not only increases the possibility of backflow from the output to the input due to the increased number of delivery tubes and syringes, but there is also a danger that fluid from the first syringe may be pulled into the tubing associated with the second syringe or the second syringe itself and undesirably mix with the second fluid. If one or the other of the syringes or its associated tubing is filled in whole or in part with air, air could also possible be introduced into the syringe being used for a fluid injection procedure which could result in an air embolism. As indicated previously, the two fluids typically used in imaging procedures are contrast and saline. The syringe associated with the contrast fluid may operate at substantially higher pressures than the saline syringe. Without adequate structure in place in the fluid path, these two fluid fluids could undesirably mix in the fluid path during a fluid injection procedure or post the fluid injection procedure due to the pressure gradient between the two syringes. As is known in the imaging field, saline is normally used during a body pre-scan prior to the injection of contrast. This pre-scan is used for digital subtraction or superposition of images. In order to prevent the degradation of the final image, the introduction of contrast into the saline portion of the fluid path during the pre-scan procedure should be prevented. However, in fluid delivery systems including conduits for both saline and contrast, the likelihood of mixture of the two fluids is somewhat high due to the configuration of the fluid delivery system.

In order to include multiple syringes each having a delivery tube in a fluid delivery system, medical connectors are typically used to direct fluid flow from multiple syringe delivery tubes into a single output delivery tube which carries fluid into a patient via catheter. Such connectors are well-known for connecting the distinct fluid delivery tubes. For example, a first delivery tube for a first fluid such as saline and a second delivery tube for a second fluid such as contrast media may be placed in fluid communication with one another through the use of a Y-connector. In such a typical system, the Y-connector is commonly used to connect the saline delivery tube and the contrast delivery tube to a single output delivery tube ultimately connected to a catheter inserted into a patient. In such an arrangement, one or more check valves are provided in the fluid path to prevent mixing of saline and contrast. Typically, at least one check valve is provided to isolate the saline fluid path from the contrast fluid path to prevent contrast mixing with the saline in the saline side of the fluid path. The position of this check valve in the fluid delivery system thus determines if any contrast will be mixed with the saline and delivered to the patient. However, the presence of this check valve further prevents patency checks from being accomplished with the saline syringe injector. Thus, it desirable to provide a patency-compatible check valve in such a fluid delivery system which is normally closed but which may be actuated to permit reverse fluid flow for patency checks.

### SUMMARY OF THE INVENTION

In one embodiment of a patency check compatible check valve described in detail herein, the check valve comprises a housing body, a valve member associated with the housing body, and an actuator operatively connected to the valve member. The housing body defines a flow passage, an inlet port communicating with the flow passage, and an outlet port communicating with the flow passage. The housing body further comprises a seal seat in the flow passage between the inlet port and outlet port. The valve member is disposed in the flow passage and is adapted to engage the seal seat. The valve member comprises a closed position wherein the valve member engages the seal seat and prevents fluid communication between the inlet port and outlet port and an open position permitting fluid flow from the inlet port to the outlet port in response to fluid flow in the inlet port. The actuator is adapted to place the valve member in an override position permitting bi-directional fluid flow through the flow passage.

The valve member is desirably fluid flow responsive to reverse fluid flow in the outlet port to engage the seal seat and attain the closed position. In one form, the actuator may comprise a lever coupled to the valve member and adapted to move the valve member to the override position. The lever may comprise an eccentric cam coupled with the valve member such that actuation of the lever causes the eccentric cam to move the valve member to the override position. Additionally, the valve member may comprise a plunger with a seal portion adapted to engage the seal seat and the lever may comprise an eccentric cam such that actuation of the lever causes the eccentric cam to move the valve member to the override position.

In another form, the valve member may comprise a disk member biased into engagement with the seal seat. The actuator may comprise a hand-actuated plunger coupled to the disk member such that actuation of the plunger overcomes the biasing force applied to the disk member to place the disk member in the override position. The disk member may be biased into engagement with the seal seat by a biasing member, such as a spring as an example.

In yet another form, the valve member may comprise a hollow member defining an internal flow passage in fluid communication with the flow passage of the housing body and at least one side port which may communicate with the internal flow passage. In this embodiment, fluid flow in the inlet port causes deformation of the hollow member to permit fluid communication between the inlet port and outlet port and place the hollow member in the open position. The deformation typically occurs along a longitudinal axis of the hollow member. The hollow member may be resiliently deformable such that upon ceasing of fluid flow in the inlet port the hollow member resiliently returns to the closed position. The seal seat may comprise an internal portion of the housing body in this embodiment. The actuator may be coupled to the hollow member to move the hollow member axially in the flow passage of the housing body to the override position wherein the at least one side port is in fluid communication with the inlet port. In this configuration, the actuator may comprise a plunger associated with an end of the hollow member such that actuation of the plunger imparts axial movement to the hollow member. The housing body may comprise a plurality of inlet ports and the hollow member may be associated with each inlet port to form the closed position therewith. In one specific form, the hollow member may be tubular shaped.

The patency check compatible check valve according to another embodiment comprises a housing body defining a flow passage and a cantilever member disposed in the flow passage. The housing body, as described previously, may define a flow passage, an inlet port communicating with the flow passage, and an outlet port communicating with the flow passage. The housing body further comprises a seal seat in the flow passage between the inlet port and outlet port. The cantilever valve member is adapted to engage the seal seat, and comprises a closed position wherein the cantilever valve member engages the seal seat and prevents fluid communication between the inlet port and outlet port and an open position permitting fluid flow from the inlet port to the outlet port in response to fluid flow in the inlet port.

The seal seat may again comprise an internal portion of the housing body. In one form, the cantilever valve member may comprise a resilient leaf spring. The housing body may alternatively comprise two inlet ports and the cantilever valve member may be fluid flow responsive to fluid flow such that fluid flow in one of the two inlet ports causes the cantilever valve member to form the closed position with the other inlet port.

The patency check compatible check valve according to further embodiment comprises a housing body and a valve member capable of having multiple states. The housing body defines a flow passage, a first inlet port communicating with the flow passage, a second inlet port communicating with the flow passage, and an outlet port communicating with the flow passage. The first and second inlet ports each comprise an inlet port member extending into the flow passage from opposing sides. The valve member is disposed in the flow passage and comprises opposing recesses receiving the opposing first and second inlet port members. The valve member is adapted to form a fluid seal with the opposing first and second inlet port members. The valve member is generally fluid flow responsive to fluid flow in one or both of the first and second inlet ports to form multiple states. These multiple states include at least: a first state wherein fluid communication between the first inlet port and the outlet port is present while a fluid seal is present between the second inlet port and the outlet port; a second state wherein fluid communication between the second inlet port and the outlet port is present while a fluid seal is present between the first inlet port and the outlet port; and a third state wherein fluid communication is at least partially present between both the first inlet port and the second inlet port and the outlet port.

In one form, the valve member may be cylindrical shaped and the opposing recesses are desirably defined in opposite ends of the cylindrical valve member. The first and second inlet ports may be segmented. Such segmentation may be in a form wherein the first and second inlet ports are each formed as a slotted dome.

The patency check compatible check valve according to a still further embodiment comprises a housing body, a valve member, and a bypass actuator. As described previously, the housing body typically defines a flow passage, an inlet port communicating with the flow passage, and an outlet port communicating with the flow passage. The housing body typically further comprises a seal seat in the flow passage between the inlet port and outlet port. The valve member is disposed in the flow passage and is adapted to engage the seal seat. The valve member comprises a closed position wherein the valve member engages the seal seat and prevents fluid communication between the inlet port and outlet port and an open position permitting fluid flow from the inlet port to the outlet port in response to fluid flow in the inlet port. The bypass actuator defines, at least in part, a bypass passage and is adapted to selectively place the inlet port in fluid communication with the outlet port. The bypass actuator has a first position wherein fluid flow through the bypass passage to the outlet port is prevented and a bypass position wherein fluid communication is enabled between the inlet port and the outlet port via the bypass passage.

The valve member may comprise a hollow member defining an internal flow passage in fluid communication with the flow passage of the housing body. In this embodiment, fluid flow in the inlet port causes deformation of the hollow member to permit fluid communication between the inlet port and outlet port and place the hollow member in the open position. The deformation typically occurs along a longitudinal axis of the hollow member. The hollow member may be resiliently deformable such that upon ceasing of fluid flow in the inlet port the hollow member resiliently returns to the closed position. The seal seat may comprise an internal portion of the housing body in this embodiment. In one specific form, the hollow member may be tubular shaped.

The housing body may comprise a plurality of inlet ports and a valve member may be associated with each inlet port to form the closed position therewith. The valve member may comprise a disk member adapted to seat against the seal seat. The bypass actuator may be adapted for rotational movement to select between the first position and the bypass position.

The bypass actuator may comprise a plurality of bypass passages to enable fluid communication between the inlet port and the outlet port via multiple bypass passages. The bypass actuator may be adapted for rotational movement to select between the first position and the bypass position.

In a particular form, the bypass actuator may comprise a bypass plunger disposed in a cavity defined by the housing body. In this form, in the first position, the bypass plunger prevents fluid flow through the bypass passage and in the bypass position at least in part defines the bypass passage such that fluid communication is enabled between the inlet port and the outlet port. The first position may comprise a raised position of the bypass plunger in the cavity and the bypass position may comprise a depressed position of the bypass plunger in the cavity. The bypass plunger may comprise a plunger head seated in the cavity and a plunger stem extending outward from the housing body. A bottom side of the plunger head typically defines a greater fluid contacting surface area than a top side of the plunger head such that reverse fluid flow in the outlet port automatically returns the bypass plunger to the first position

Further details and advantages will become clear upon reading the following detailed description in conjunction with the accompanying drawing figures, wherein like parts are identified with like reference numerals throughout.

### BRIEF DESCRIPTION OF THE DRAWINGS

FIG. 1 is a schematic view of a fluid path for sequential fluid injection procedures involving two fluids.

FIG. 2 is a schematic view of a fluid path for simultaneous fluid injection procedures involving two fluids.

FIG. 3 is a perspective view of a first embodiment of a patency check compatible check valve for use in the fluid paths of FIGS. 1-2.

FIG. 4 is an exploded perspective view of the check valve of FIG. 3.

FIG. 5 is a top plan view of the check valve of FIG. 3 in a normal state.

FIG. 6 is a cross-sectional view taken along lines 6-6 in FIG. 5.

FIG. 7 is a top plan view of the check valve of FIG. 3 in an override or bypass state.

FIG. 8 is a cross-sectional view taken along lines 8-8 in FIG. 7.

FIG. 9 is a perspective detail view of the mechanical components permitting operation of the check valve of FIG. 3 between the normal state and the override state.

FIG. 10 is a side view of a second embodiment of the patency check compatible check valve shown associated with a fluid injection syringe or pressurizing device.

FIG. 11 is an exploded perspective view of the check valve of FIG. 10.

FIG. 12 is a top plan view of the check valve of FIG. 10.

FIG. 13 is a transverse cross-sectional view taken along lines 13-13 in FIG. 12 and showing the check valve of FIG. 10 in the normal state.

FIG. 14 is a transverse cross-sectional view taken along lines 14-14 in FIG. 12 and showing the check valve of FIG. 10 and in the override state.

FIG. 15 is perspective view of a third embodiment of the patency check compatible check valve.

FIG. 16 is an exploded perspective view of the check valve of FIG. 15.

FIG. 17 is a horizontal cross-sectional view of the check valve of FIG. 15 taken along lines 17-17 in FIG. 15 and showing the check valve in the normal state.

FIG. 18 is a horizontal cross-sectional view of the check valve of FIG. 15 shown in the override state.

FIG. 19 is a perspective view of an actuator associated with the check valve of FIG. 15 and adapted to place the check valve in the override state.

FIG. 20 is a perspective view showing the actuator of FIG. 20 interfacing with a valve member of the check valve of FIG. 15.

FIG. 21 is a perspective view of a fourth embodiment of the patency check compatible check valve.

FIG. 22 is an exploded perspective view of the check valve of FIG. 21.

FIG. 23 is a horizontal cross-sectional view of the check valve of FIG. 21 shown in a normal, pre-actuated state.

FIG. 24 is a horizontal cross-sectional view of the check valve of FIG. 21 and showing operation of the check valve in dashed lines.

FIG. 25 is a perspective view of a fifth embodiment of the patency check compatible check valve.

FIG. 26 is an exploded perspective view of the check valve of FIG. 25.

FIG. 27 is a transverse cross-sectional view of the check valve of FIG. 25 shown in a first state.

FIGS. 28A-28C are transverse cross-sectional views of the check valve of FIG. 25 showing three operational states of the check valve.

FIG. 29A is a detail cross-sectional view showing the operational state of the check valve depicted in FIG. 28A.

FIG. 29B is a detail cross-sectional view showing the operational state of the check valve depicted in FIG. 28B.

FIG. 30 is a perspective view of a sixth embodiment of the patency check compatible check valve.

FIG. 31 is an exploded perspective view of the check valve of FIG. 30.

FIG. 32 is a side view of the check valve of FIG. 30 and showing a bypass actuator of the check valve in a first position.

FIG. 33 is a horizontal cross-sectional view taken along lines 33-33 in FIG. 32 and showing the check valve in the normal state.

FIG. 34 is a side view of the check valve of FIG. 30 and showing the bypass actuator of the check valve in a second or bypass position.

FIG. 35 is a horizontal cross-sectional view taken along lines 35-35 in FIG. 34 and showing the check valve in the override or bypass state.

FIG. 36 is a perspective view of a seventh embodiment of the patency check compatible check valve and omitting an optional dome protective cap for clarity.

FIG. 37 is an exploded perspective view of the check valve of FIG. 36.

FIG. 38 is a perspective view of a housing body associated with the check valve of FIG. 36.

FIG. 39 is a perspective view of the check valve of FIG. 36 showing a bypass actuator associated with the housing body of FIG. 38 and in a first, raised position in the housing body.

FIG. 40 is a perspective view of the check valve of FIG. 36 showing the bypass actuator associated with the housing body of FIG. 38 and in a second, depressed bypass position in the housing body.

FIG. 41 is a perspective view of the bypass actuator associated with the check valve of FIG. 36.

FIG. 42 is a perspective view of the bypass actuator of FIG. 41 according to an alternative embodiment.

FIG. 43A is a transverse cross-sectional view taken along lines 43A-43A in FIG. 36 and showing the check valve in the normal state.

FIG. 43B is a transverse cross-sectional view taken along lines 43B-43B in FIG. 36 and showing the check valve in a normal state.

FIG. 44A is a transverse cross-sectional view similar to FIG. 43A but showing the check valve in the override or bypass state with the bypass actuator in the second, depressed bypass position in the housing body.

FIG. 44B is a transverse cross-sectional view similar to FIG. 43B but showing the check valve in the override or bypass state with the bypass actuator in the second, depressed bypass position in the housing body.

FIG. 45 is a transverse cross-sectional view taken along lines 45-45 in FIG. 36 and showing the check valve in the normal state.

FIG. 46 is a perspective view of an eighth embodiment of the patency check compatible check valve and showing the check valve in the normal state.

FIG. 47 is a perspective view of the patency check compatible check valve of FIG. 46 and showing the check valve in the override or bypass state.

FIG. 48 is an exploded perspective and cross-sectional view of the check valve of FIG. 46.

FIG. 49 is a transverse cross-sectional view taken along lines 49-49 in FIG. 46.

FIG. 50 is a transverse cross-sectional view taken along lines 50-50 in FIG. 49.

FIG. 51 is a transverse cross-sectional view taken along lines 51-51 in FIG. 46.

FIG. 52 is a transverse cross-sectional view taken along lines 52-52 in FIG. 47.

FIG. 53 is a transverse cross-sectional view taken along lines 53-53 in FIG. 52.

FIG. 54 is a transverse cross-sectional view taken along lines 54-54 in FIG. 47.

### DESCRIPTION OF THE PREFERRED EMBODIMENTS

For purposes of the description hereinafter, spatial orientation terms, if used, shall relate to the referenced embodiment as it is oriented in the accompanying drawing figures or otherwise described in the following detailed description. However, it is to be understood that the embodiments described hereinafter may assume many alternative variations and configurations. It is also to be understood that the specific devices illustrated in the accompanying drawing figures and described herein are simply exemplary and should not be considered as limiting.

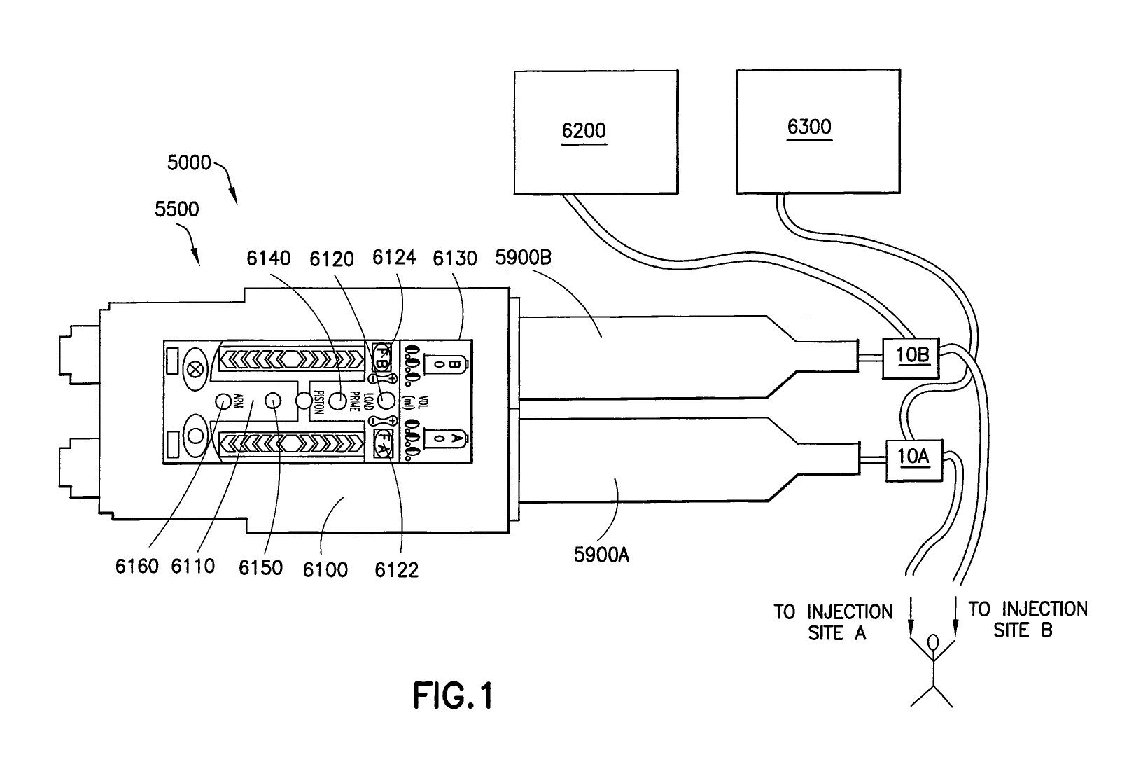

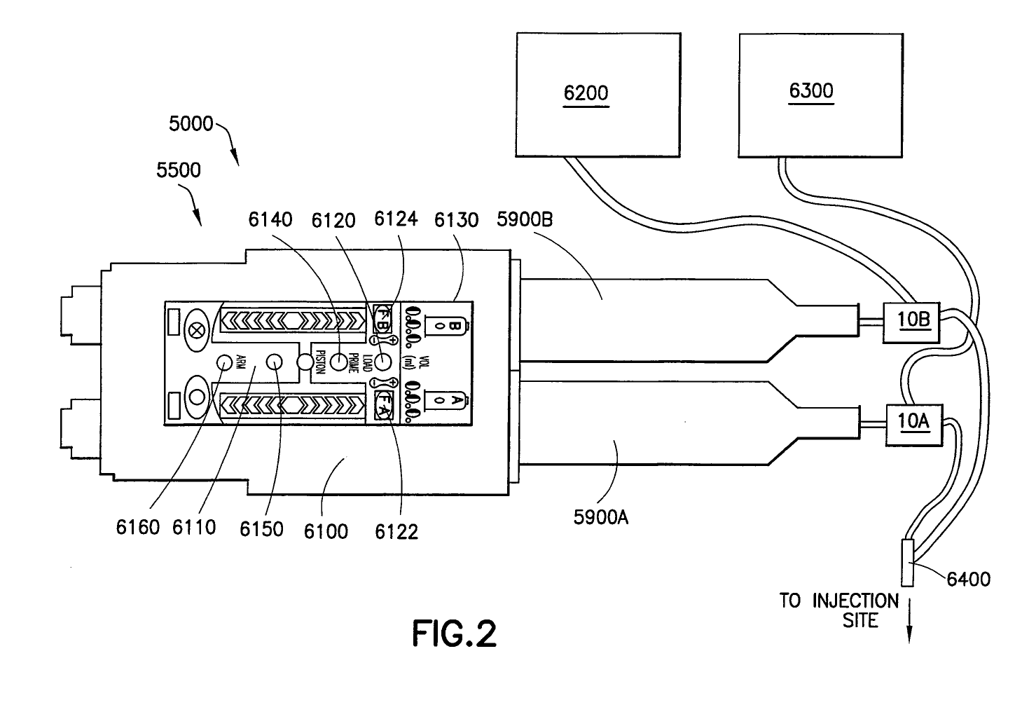

Referring to FIGS. 1-2, a patency check compatible check valve 10, the details of several embodiments of which are set forth herein, is illustrated as part of a multi-syringe fluid injector system 5000 as described in application Ser. Nos. 10/722,370, filed Nov. 25, 2003, and 10/159,592 filed May 30, 2002 the disclosures of which are incorporated by reference herein. In the foregoing application Ser. Nos. 10/722,370 and 10/159,592 an injector 5500 and graphical user interfaces for control thereof are disclosed. In one exemplary application, the injector 5500 and associated user-interface control devices are used in the computerized tomography (CT) environment. In a typical CT environment, a control unit (not shown) for control of injector 5500 is placed in a control room which is shielded from radiation used to produce the CT scan. Injector 5500 is positioned within a scan room with the CT scanner and a scan room control unit in communication with injector 5500 and in communication with the control room unit. The scan room control unit can duplicate some or all of the control features found on the control room unit as known in the art. Moreover, the scan room control unit can include injector control features in addition to those found on the control room unit as known in the art. Other control units such as a handheld control unit can also be provided as known in the art.

In a typical procedure, the operator of the CT procedure first programs the protocol for the injection procedure using the control room unit and, typically, a graphical user interface (not shown) for the control room unit. In a typical CT procedure, the control system of injector 5500 desirably includes three modes of injection selectable by the operator. These modes of operation include a mode for sequential injection from syringes 5900A and 5900B, a mode for simultaneous injection from syringes 5900A and 5900B into a single injection site, and a mode for simultaneous injection from syringes 5900A and 5900B into different injection sites. In the case of a sequential injection, a fluid can be injected from only one of syringe 5900A or 5900B at a time. For example, syringe 5900A may contain contrast medium (hereinafter “contrast”), while syringe 5900B may contain a flushing fluid such as saline, which may be sequentially injected into a patient using a variety of protocols as known in the art. An example of a fluid path for sequential injection is illustrated in FIG. 2. In FIG. 2, tubing from each of syringes 5900A and 5900B come together via a T-connector 6400 for fluid connection to the injection site in the patient. A plurality of phases of sequential injection may be entered using control room graphic user interface (not shown) as detailed in application Ser. Nos. 10/722,370 and 10/159,592. During simultaneous injection into a single site (using, for example, the fluid path of FIG. 2), syringe 5900A may, for example, be loaded or filled with contrast, while syringe 5900B may, for example, be loaded with a diluent or flushing fluid such as saline. In this mode, contrast or other fluid in syringe 5900A may, for example, be diluted or mixed with fluid in syringe 5900B to a desired concentration by simultaneous injection from syringe 5900A and, 5900B as programmed by the operator. In the case of a simultaneous injection to different injection sites (see FIG. 1), syringe 5900A and syringe 5900B may, for example, both be filled with the same injection fluid (for example, contrast). Injection of the contrast at two different sites, as opposed to a single site may, for example, enable delivery of a desired amount of contrast to a region of interest at a lower flow rate and a lower pressure at each site than possible with injection into a single site. For example, half the contrast desired for delivery to the heart of patient P heart may be injected into a vein on each arm of the patient P (see FIG. 1), rather than injection of the entire amount into a single injection site on one of the his or her arms. The lower flow rates and pressures enabled by simultaneous injection into multiple sites may, for example, reduce the risk of vascular damage and extravasation.

After setting the desired protocols at the control room unit in any of the above injection modes, the operator typically enters the scan room for final preparations of injector 5500 and/or final preparations of the patient. In the embodiments of FIGS. 1-2, the scan room control unit is part of or in incorporated into injector 5500 with a control/display GUI or interface 6100 positioned on an upper side of injector 5500. Incorporating the scan room control unit into injector 5500 can, for example, reduce the use of space within the scan room as compared to a separate control unit. The control room interface (not shown) typically includes a lock protocol function which may, for example, be a button, micro-switch, or touch screen area activated by the operator to “lock” the protocol. Subsequent editing of the injection protocol preferably deactivates the protocol function lock. Alternatively, activation of the protocol lock can prevent editing of the set protocol until the protocol function lock is deactivated. Activation and deactivation of the protocol function lock preferably changes the state, for example, activates and deactivates, respectively, an indicator such as a light 6110 on interface 6100. When activated, indicator light 6110 ensures the operator that another person has not altered the set protocols while the operator was in the scan room. In that regard, editing of the set protocol is associated with deactivation of the protocol function lock, and deactivation of protocol function lock 6010 results in a change of state, for example deactivation, of indicator light 6110. The protocol function lock can, for example, lock out further protocol editing and be password encoded for extra assurance that undesired protocol changes are not entered.

After connection of empty syringes 5900A and 5900B to injector 5500, the configurations of syringes 5900A and 5900 are preferably sensed by the injector 5500 and the injector 5500 may execute certain procedures such auto docking or auto-engaging as well as auto-advancing as described in application Ser. Nos. 10/722,370 and 10/159,592. Injector system 5000 is now ready for filling of empty syringes 5900A and 5900B. In the illustrated embodiments, syringes 5900A and 5900B are in fluid connection with sources or reservoirs of injection fluid, 6200 and 6300, respectively. For example, source 6200 may be a reservoir of contrast while source 6300 may be a reservoir of a flushing or diluting fluid such as saline. A valve system which desirably includes check valves 10A, 10B, one or both of which may be patency check compatible, is provided to control fluid flow to prevent cross contamination between patients when, for example, sources 6200 and/or 6300 are used with multiple patients.

Auto-loading and/or auto-priming can begin automatically upon setting of protocols as described application Ser. Nos. 10/722,370 and 10/159,592. Alternatively, auto-loading can be manually initiated, at least in part, by the operator via activation of an auto-load switch 6120 as well as fill switches or buttons 6122 and 6124 for each of syringes 5900A and 5900B, respectively, on scan room control unit interface 6100. A display area 6130 of interface 6100 can, for example, include a numeric display as well as a graphical display of the amount of fluid in each of syringes 5900A and 5900B. Different colors may be used to denote the different syringes and the different fluids therein. In the auto loading and/or auto priming process display area 6130 as well as a display area of the control unit interface (not shown) indicate that auto-loading has not yet been initiated and each of syringes 5900A and 5900B is indicated to be empty (0 ml volume). Display area 6130 after activation of the auto-load switch will indicate the amount of saline that will be loaded into syringe 5900B and the amount of contrast that will be loaded into syringe 5900A. Upon confirmation/acceptance of the fill volumes, the operator activates each of fill switches or buttons 6122 and 6124 to begin loading of contrast and saline into syringes 5900A and 5900B, respectively. Upon activation of an auto prime switch or button 6140, a preselected amount of contrast, for example 1 ml, is injected from syringe 5900A, and a preselected amount of saline, for example 4 ml, is injected from syringe 5900B to prime the fluid path and tubing set. The tubing can now be connected to a patient catheter.

Syringes 5900A and 5900B are now in a state to commence injection. Preferably, injector 5500 requires the operator to perform a check for air in the fluid path as known in the art. In that regard, the injector system can prevent injection until an air check confirmation button or switch 6150 on scan room unit interface 6100 is activated. After arming injector 5500 by, for example, activating an arm switch or button on interface 6000 or a similar arm switch or button 6150 on interface 6100, the injection can be initiated. As know in the art, arming the injector 5500 can initiate a number of self or internal tests and state checks to ensure that injector 5500 is ready for injection. One of these checks desirably includes a patency check. As described previously, a patency check is conducted by actuating the syringe injector so that the syringe plunger is momentarily retracted until blood or another body fluid is detected in the tubing of the fluid path, thereby confirming correct catheter placement in a blood vessel, such as an artery or vein. As further described previously, conventional check valves prevent this procedure from being conducted due to their one-directional flow path. However, the use of check valves 10A, 10B, one or both of which may be patency check compatible, in association with the fluid path of syringes 5900A and 5900B allows a patency check to accomplished with either syringe 5900A, 5900B. In conventional practice, saline-containing syringe 5900B is typically used for a patency check and, accordingly, check valve 10B is desirably patency check compatible and may be one of the embodiments of patency check compatible check valve 10 described hereinafter. If desired check valve 10A may be a conventional one-directional check valve as is known in the art.

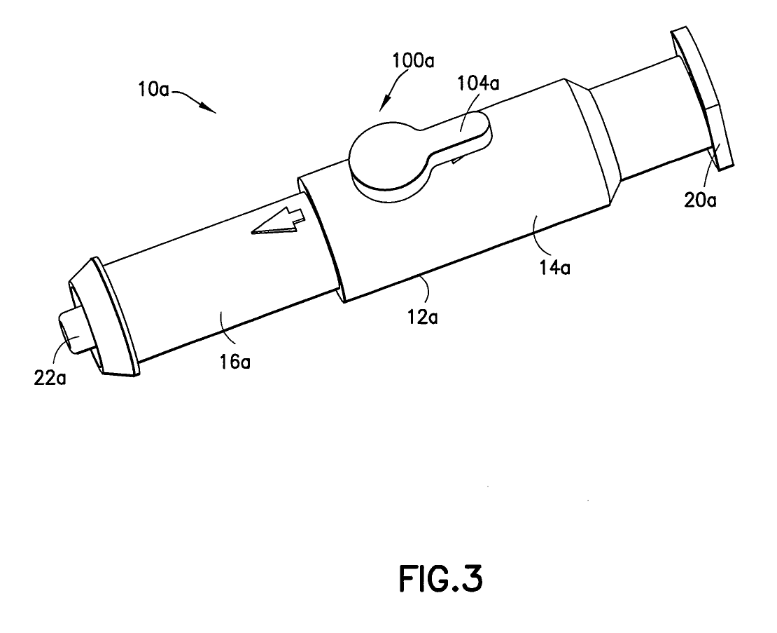

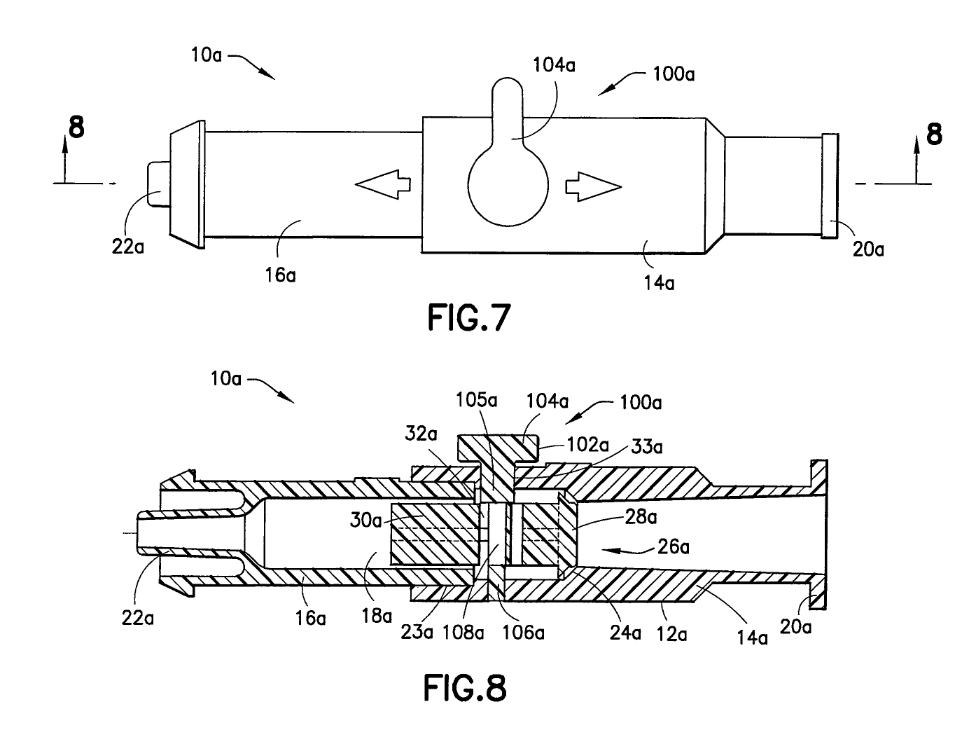

In view of the foregoing, it will be appreciated that each embodiment of patency check compatible check valve 10 to be discussed hereinafter may be used as check valve 10A and/or 10B in the fluid injector system 5000 of FIGS. 1-2. Each respective embodiment of patency check compatible check valve 10 discussed herein is identified with a lower case alphanumeric designation in explaining the various embodiments. Accordingly, a first embodiment of patency check compatible check valve 10a (hereinafter “check valve 10a”) is shown in FIGS. 3-9. Check valve 10a comprises a housing body 12a which may be a unitary body or, as illustrated, comprised of two joined housing portions, including a first housing portion 14a and a second housing portion 16a that are assembled to form housing body 12a. First and second housing portions 14a, 16a of housing body 12a, when secured together, define a flow passage 18a for fluid flow through the housing body 12a. First and second housing portions 14a, 16a respectively define an inlet port 20a and an outlet port 22a which communicate with flow passage 18a. Inlet port 20a and outlet port 22a may be formed with standard luer connection configurations. For example, inlet port 20a may be formed with a standard threaded female luer connection and outlet port 22a may be formed with a standard threaded male luer connection or this configuration may be reversed. First and second housing portions 14a, 16a may be joined by conventional joining techniques known in the medical art. For example, second housing portion 16a be inserted and maintained in first housing portion 14a via frictional engagement with this frictional engagement secured by adhesive, solvent, laser, or ultrasonic bonding methods along an engagement interface 23a between first and second housing portions 14a, 16a.

First housing portion 14a defines a seal seat 24a internally within flow passage 18a that is generally circular in configuration but may take other suitable forms. Seal seat 24a is provided in flow passage 18a between inlet port 20a and outlet port 22a. Generally, seal seat 24a is a tapered surface against which a valve element or structure may make a sealing connection or engagement to regulate fluid flow through flow passage 18a. A valve member 26a is disposed in the flow passage 18a between inlet port 20a and outlet port 22a and is tapered desirably at least in part in a corresponding manner to seal seat 24a to mate therewith. Accordingly, valve member 26a is adapted to engage and seal against seal seat 24a and provide a substantially fluid tight seal therewith. Valve member 26a is generally operable as described herein to have at least two flow states including a normally closed position or state wherein the valve member 26a engages seat 24a. In the closed position or state, valve member 26a engages seat 24a but is operable in response to fluid flow in inlet port 20a to move to an open position or state permitting one-directional fluid flow from inlet port 20a to outlet port 22a thereby allowing fluid flow to pass through flow passage 118a from the inlet port 20a to the outlet port 22a. When fluid flow in inlet port 20a ceases, valve member 26a is adapted to return to the normally closed position or state in engagement with seal seat 24a. A second or override position or state of valve member 26a, also referred to as a bypass position or state herein, occurs when valve member 26a is placed and maintained in the open position or state unseated from seal seat 24a which permits bi-directional fluid flow through the flow passage 18a thereby allowing check valve 10a to be used for patency checks in, for example, the fluid injector system 5000 shown in FIGS. 1-2.

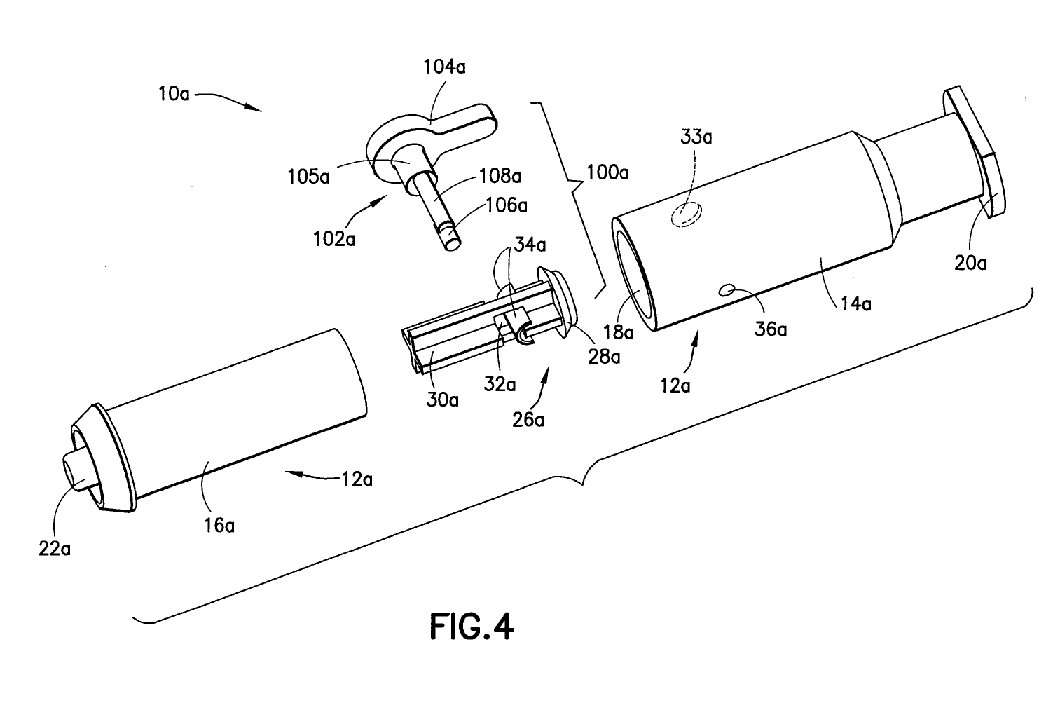

In this embodiment, valve member 26a is generally plunger-shaped and comprises a disk-shaped distal seal portion 28a adapted to engage in corresponding manner with seal seat 24a and a proximal plunger portion 30a extending from seal portion 28a. Valve member 26a may be integrally formed of thermoplastic material such polypropylene, polyethylene, or polycarbonate but seal portion 28a desirably has sufficient resiliency or compliancy to form a generally fluid tight seal with seal seat 24a when engaged therewith. If desired, seal portion 28a of valve member 26a may be formed of a different material from plunger portion 30a, such as a sealing compliant material as, for example, rubbers, thermoplastic elastomers, or silicone, and be joined by the conventional joining techniques identified previously to plunger portion 30a which will serve as a stiffening and control element associated with valve member 26a. Accordingly, valve member 26a may be singular structure with seal portion 28a and plunger portion 30a integrally formed or, alternatively, seal portion 28a may be formed separately from plunger portion 30a and secured in permanent or semi-permanent fashion with plunger portion 30a such as by an adhesive or any of the conventional joining techniques identified previously. In the foregoing bifurcated arrangement, plunger portion 30a is desirably formed of a harder plastic material such as polypropylene, polyethylene, or polycarbonate and seal portion 30a is desirably formed of a more resilient, compliant material for effecting a seal with seal seat 24a such as such as rubbers, thermoplastic elastomers, or silicone as examples. As shown in FIG. 4, for example, plunger portion 30a may be segmented such as having an X-shaped transverse cross section for increased strength and rigidity. However, this configuration is only exemplary and should not be considered as limiting. Plunger portion 30a further defines a control aperture 32a, a boss aperture 33a, and a biasing plate 34a provided just distal or in front of control aperture 32a.

An override or bypass actuator 100a is operatively connected to valve member 26a and, in particular, with plunger portion 30a of the valve member 26a. Actuator 100a is adapted to place valve member 26a in the override or bypass position or state discussed previously thereby permitting bi-directional fluid flow through flow passage 18a. In the present embodiment, actuator 100a comprises a lever member 102a formed with a lever handle 104a at one end connected to a boss 105a and a lever shaft 106a at the opposite and an eccentric cam lobe or shaft 108a connecting the boss 105a on the lever handle 104a and the lever shaft 106a. Lever handle boss 105a is seated in boss aperture 33a and eccentric cam shaft 108a is seated in control aperture 32a in plunger portion 30a to operatively associate actuator 100a with valve member 26a. In this orientation, eccentric cam shaft 108a is positioned so that at least a portion of the length of the eccentric cam shaft 108a is in operative engagement with at least a portion of the rear or proximal side of biasing plate 34a associated with plunger portion 30a. Additionally, lever shaft 106a is journaled for rotation in a side aperture 36a in the first or inlet housing portion 14a of housing body 102a. The operative engagement of eccentric cam shaft 108a in control aperture 32a as well as the rotational motion afforded by the rotational connection between lever shaft 106a and housing body 12a allows rotational movement inputs to lever handle 104a to be transmitted to plunger portion 32a via eccentric cam shaft 108a which translates into axial movement of valve member 26a. This axial movement places valve member 26a in the override or bypass position or state permitting bi-directional fluid flow through flow passage 18a. In the normally closed position or state of valve member 26a, the orientation of eccentric cam shaft 108a in control aperture 32a and the interference contact between eccentric cam shaft 108a and biasing plate 34a provides sufficient tolerance to allow valve member 26a to unseat from seal seat 24a when sufficient fluid flow is present in inlet port 20a.

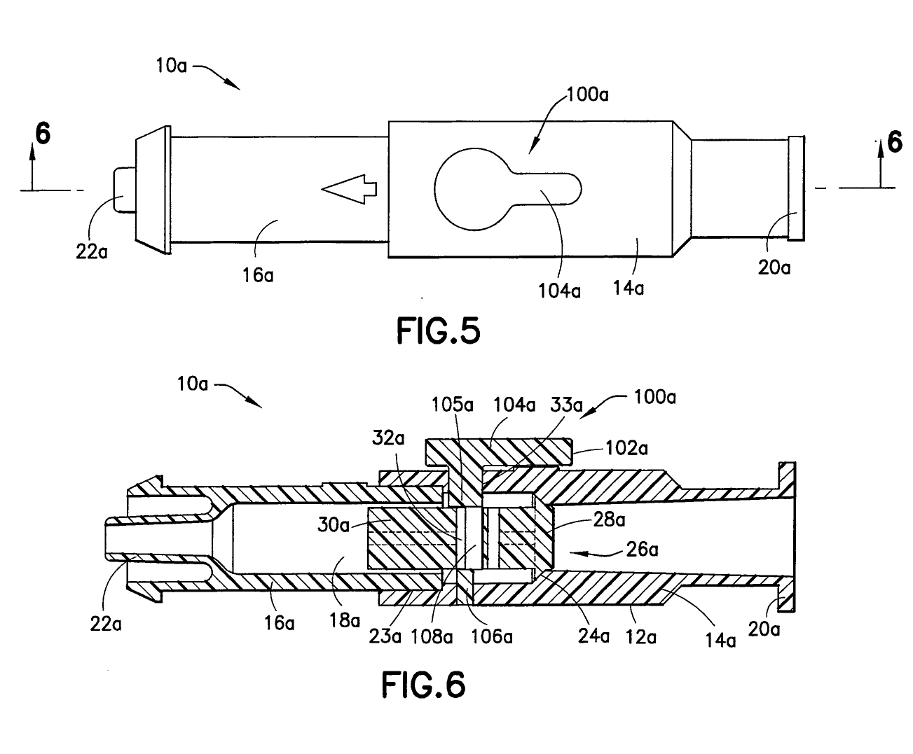

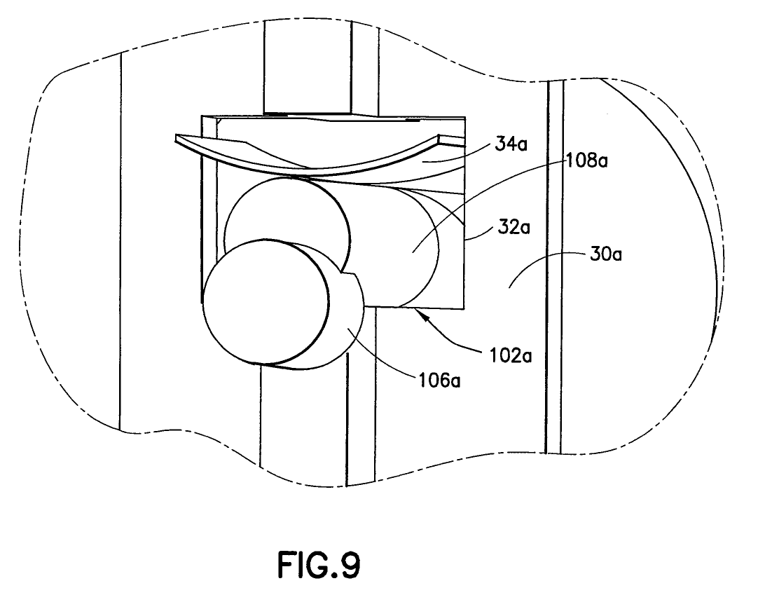

The normally closed position of valve member 26a is shown in FIG. 6 and the override or bypass position of valve member 26a is shown n FIG. 8. In the normally closed position of valve member 26a, eccentric cam shaft 108a is disposed in contact with biasing plate 34a on one side thereof and the control aperture 32a on the other (See FIG. 9). Additionally, lever shaft 106a is journaled for rotation in side aperture 36a in the first housing portion 14a of housing body 102a as indicated previously. A schematic depiction of the location and orientation of eccentric cam shaft 108a in control aperture 32a and the resulting relative orientation of lever shaft 106a is provided in FIG. 9. In this closed orientation, fluid flow in inlet port 20a of first housing portion 14a of housing body 12a applies pressure against valve member 26a and, in particular, distal seal portion 28a thereof. This pressure force is transmitted via plunger portion 30a to biasing plate 34a which operates in a manner similar to a leaf spring. The transmitted pressure force causes biasing plate 34a to deflect about its contact point or, more particularly, contact line with eccentric cam shaft 108a. This contact line is offset from a center line or axis passing through control aperture 32a allowing biasing plate 34a to deflect about the contact line. This deflection provides sufficient tolerance for valve member 26a to unseat from seal seat 24a thereby allow fluid flow in inlet port 20a to pass through flow passage 18a to outlet port 22a defined by the second housing portion 16a of housing body 12a. This flow is limited to one-direction from inlet port 20a to outlet port 22a because, once fluid pressure is no longer present in inlet port 20a and/or reverse fluid flow is present in outlet port 22a, the “deflection” pressure force applied to biasing plate 34a is no longer present and the biasing plate 34a resiliently returns to its original condition or state and, in so doing, causes seal portion 28a of valve member 26a to reseat against seal seat 24a.

When it is desired to place valve member 26a in the override or bypass position or state, an operator rotates lever handle 104a at least 90° counterclockwise in this example. In so doing, eccentric cam shaft 108a exerts proximally directed force against plunger portion 30a of valve member 26a by virtue of its contact with plunger portion 30a in control aperture 32a in the plunger portion 30a. The rotational movement input to lever handle 104a causes eccentric cam shaft 108a to apply a camming action to plunger portion 30a moving the plunger portion 30a in a proximal or reverse axial direction in flow passage 18a. This proximal or reverse axial movement unseats valve member 26a from seal seat 24a. Once the lever handle 104a is placed in the 90° counterclockwise position, the offset orientation of the eccentric cam shaft 108a will maintain the valve member 26a in the override or bypass position or state allowing bi-directional flow through flow passage 18a. It will be appreciated that there is no restriction on the rotation of eccentric cam shaft 108a and lever handle 104a may be rotated fully to the 180° position relative to the position of lever handle 104a in the closed position of valve member 26a. In the 180° position of lever handle 104a, seal portion 28a of valve member 26a is unseated from seal seat 24a to a maximum amount or distance.

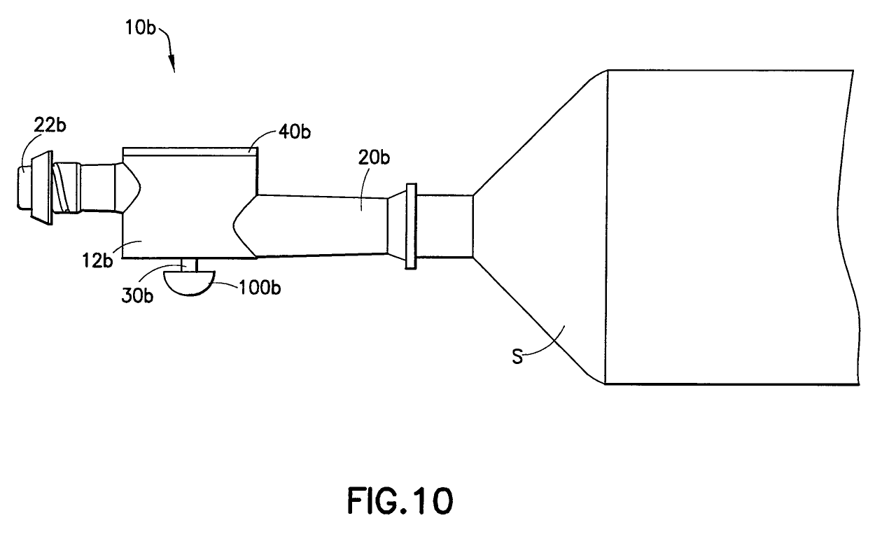

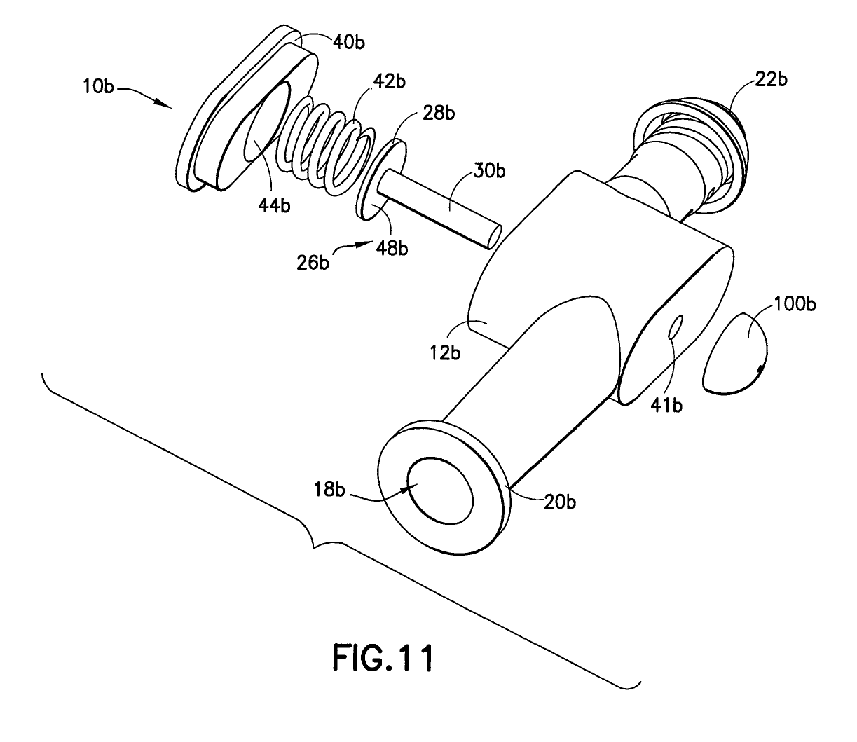

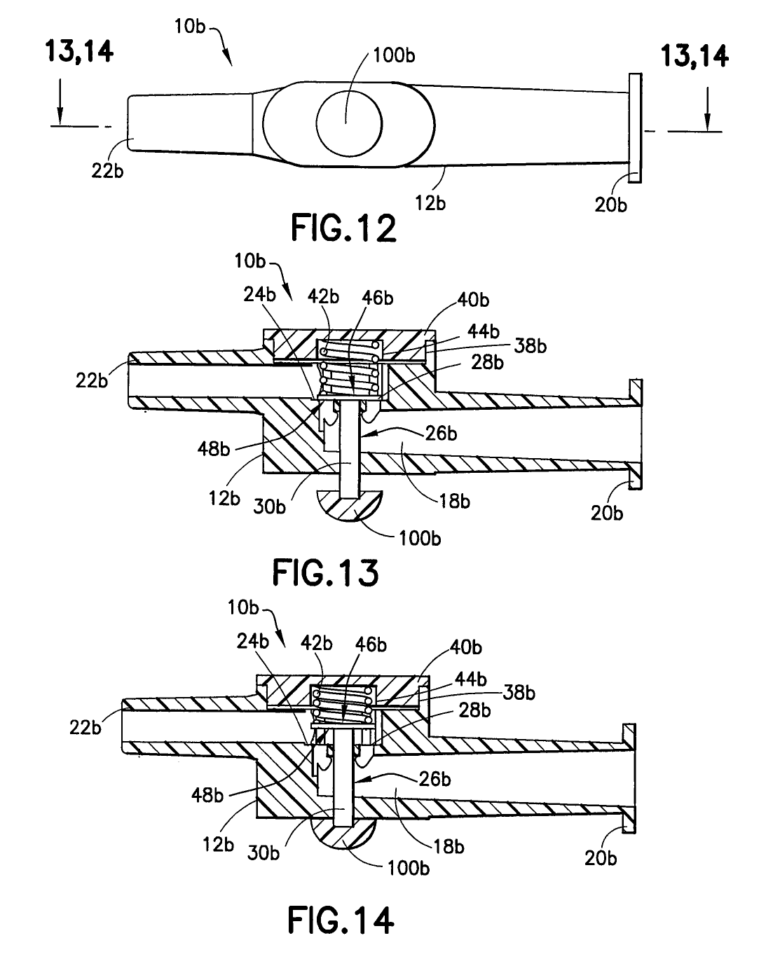

A second embodiment of check valve 10b is shown in FIGS. 10-14. Check valve 10b according to this embodiment comprises a unitary housing body 12b which defines flow passage 18b for fluid flow through the housing body 12b. Housing body 12b defines inlet port 20b and outlet port 22b which communicate with flow passage 18b in a similar manner to that described previously. As indicated previously, inlet port 20b and outlet port 22b may be formed with standard luer connections having the specific convention (or reversal thereof) described previously. Housing body 12b defines seal seat 24b internally within flow passage 18b and is again generally circular in configuration but may take other suitable forms. Seal seat 24b is provided in flow passage 18b between inlet port 20b and outlet port 22b. In this embodiment, seal seat 24b is a generally flat, annular rim surface defined by housing body 12b in flow passage 18b against which valve member 26b may make a sealing connection or engagement to regulate fluid flow through flow passage 18b. Valve member 26b is disposed within the flow passage 18b between inlet port 20b and outlet port 22b and opposite from seal seat 24b. Accordingly, valve member 26b is positioned and adapted to engage and seal against seal seat 24b and provide a substantially fluid tight seal therewith.

As with the embodiment of check valve 10a discussed previously, valve member 26b is generally operable to have at least two flow states including a normally closed position or state wherein the valve member 26b engages seat 24b. In the closed position or state valve member 26b engages seat 24b but is operable in response to fluid flow in inlet port 20b to move to an open position or state permitting one-directional fluid flow from inlet port 20b to outlet port 22b thereby allowing fluid flow to pass through flow passage 18b from inlet port 20b to outlet port 22b. When fluid flow in inlet port 20b ceases, valve member 26b is adapted to return to the normally closed position or state in engagement with seal seat 24b. A second or override position or state of valve member 26b occurs when valve member 26b is placed and maintained in the open position or state unseated from seal seat 24b which permits bi-directional fluid flow through flow passage 18b.

In this embodiment, valve member 26b is again generally plunger shaped and comprises a disk-shaped distal seal portion 28a adapted to engage seal seat 24b and a proximal plunger portion 30b extending proximally from seal portion 28b. Plunger portion 30b terminates in this embodiment in a button-shaped override or bypass actuator 100b which is desirably secured to the proximal end of plunger portion 30b by any of the conventional joining techniques identified previously. Alternatively, in this embodiment, button actuator 100b may be formed integrally with valve member 26b. The location of button actuator 100b at the proximal end of plunger portion 30b orients the button actuator 100b for access outside of housing body 12b to allow an operator to place valve member 26b in the override or bypass position or state. Valve member 26b may be assembled into housing body 12b through an access opening 38b in housing body 12b which may be enclosed by cover member 40b that is secured to housing body 12b in access opening 38b by any of the conventional joining techniques identified previously. Plunger portion 30b extends through an opening 41b in housing body 12b, with button actuator 100b thereafter being affixed to the proximal end of plunger portion 30b. A biasing member 42b, such as a coil spring, is disposed opposite seal portion 28b of valve member 26b to maintain the valve member 26b in the normally closed position as discussed further herein. As illustrated in cross section in FIGS. 13-14, biasing member 42b is secured and restrained at one end in a pocket or recess 44b in cover member 40b. Accordingly, biasing member 42b is operable between cover member 40b and a top or first side or surface 46b of seal portion 28b. A bottom or second side or surface 48b of seal portion 28b faces seal seat 24b and forms the sealing surface which engages seal seat 24b to form the generally fluid tight seal therewith. It may be desirable to place a sealing material, for example, a compliant material such as rubbers, thermoplastic elastomers, or silicone, on the bottom side or surface 48b of seal portion 28b to aid in forming the generally fluid tight seal between seal portion 28b and seal seat 24b. Again, if desired, seal portion 28b of valve member 26b may be formed of a different material from plunger portion 30b and secured in permanent or semi-permanent fashion with plunger portion 30b such as by an adhesive or any of the conventional joining techniques identified previously.

The normally closed position or state of valve member 26b is shown in FIG. 13 and the override or bypass position or state of valve member 26b is shown in FIG. 14. In the closed position, biasing member 42b provides biasing force acting against seal portion 28b and, in particular, the top side 46b of seal portion 28b to seat the bottom side 48b of seal portion 28b in engagement with seal seat 24b. The biasing force applied by biasing member 42b maintains the closed position or state of valve member 26b until sufficient fluid pressure is present in inlet port 20b of housing body 12b to unseat valve member 26b from seal seat 24b. This fluid pressure is applied to the bottom side 48b of seal portion 28b of valve member 26b and lifts valve member 26b from engagement with seal seat 24b when the fluid pressure becomes greater than the biasing force of biasing member 42b.

To place valve member 26b in the override or bypass position or state, an operator applies upward pressure to button actuator 10b. This applied pressure compresses the biasing member 42b between recess 44b in cover member 40b and the top side 46b of seal portion 28b of valve member 26b. Sufficient finger pressure must be applied to overcome the biasing force of biasing member 42b to unseat the valve member 26b from seal seat 24b. As long as sufficient pressure is applied to button actuator 10b, the biasing force of biasing member 42b is overcome and the valve member 26b is maintained in the open position or state allowing bi-directional flow through flow passage 18b. As shown in FIG. 10, check valve 10b may be associated with the discharge port of a syringe S as an exemplary application of check valve 10b in addition to use in fluid injector system 5000 discussed previously. Additionally, while check valve 10b is shown and explained in the foregoing in a downward facing orientation with button actuator 100b pointed in a downward vertical direction and, accordingly, valve member 26b oriented in the same downward direction, it will be appreciated that check valve 10b will operate in the same manner as described hereinabove if oriented in an upward vertical direction. Accordingly, the merely exemplary “top-bottom” convention assigned to seal portion 28b of valve member 26b is reversed in this alternative orientation.

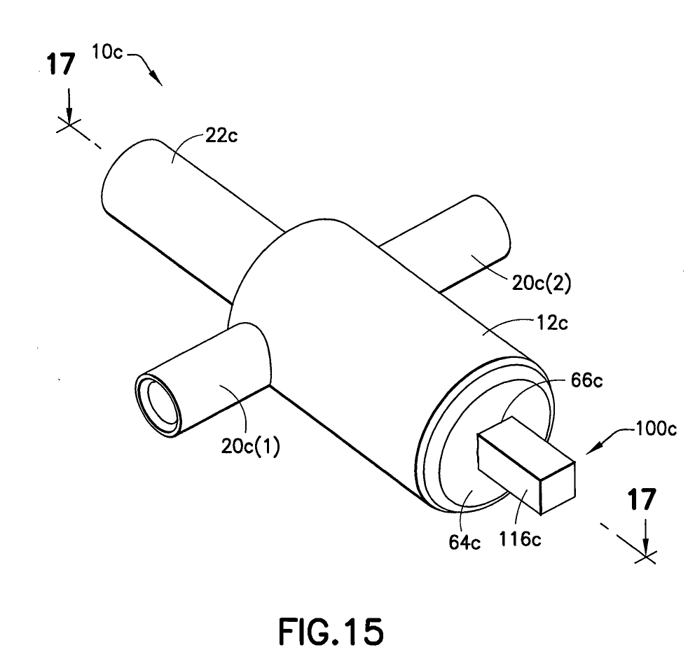

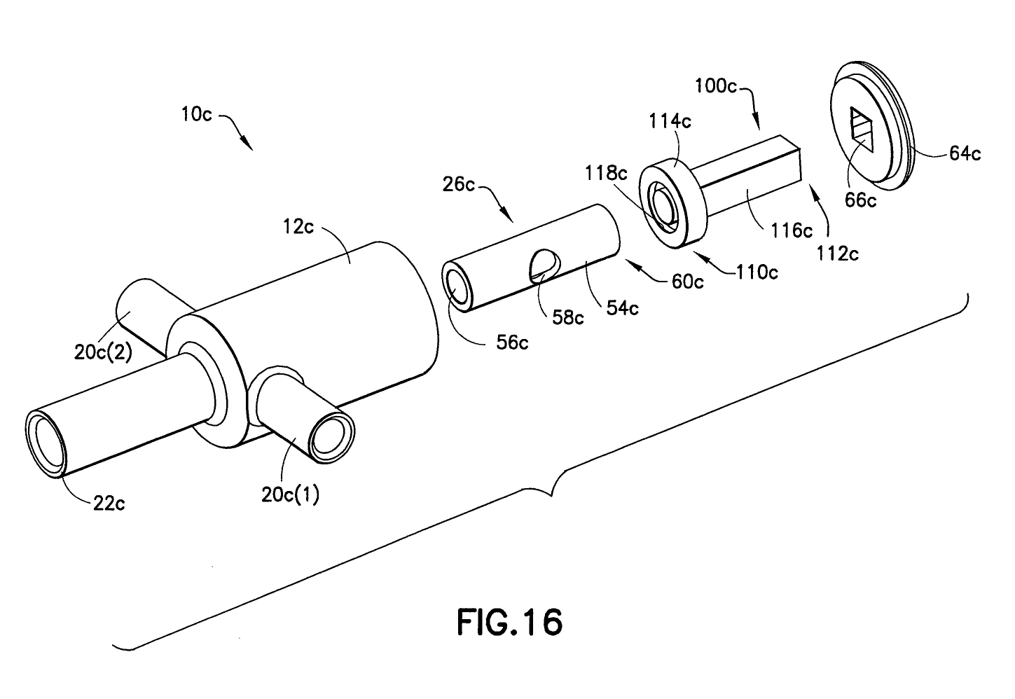

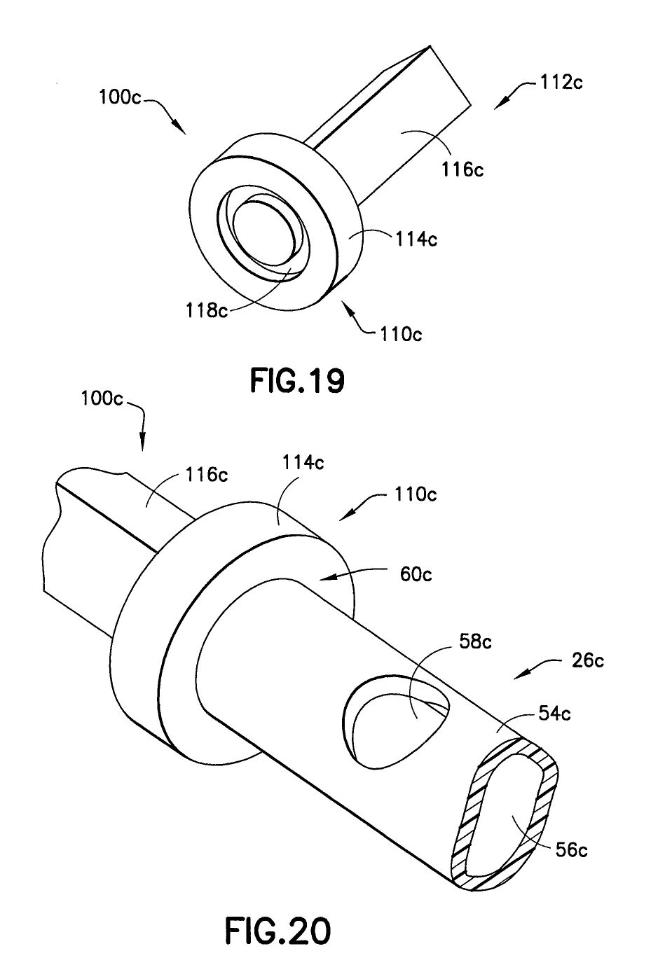

A third embodiment of check valve 10c is shown in FIGS. 15-20. Check valve 10c according to this embodiment comprises a unitary housing body 12c which defines an internal flow passage 18c for fluid flow through housing body 12c. In this embodiment, housing body 12c defines a pair of opposing first and second inlet port 20c(1), 20c(2) and an outlet port 22c which communicate with flow passage 18b. Inlet ports 20c(1), 20c(2) are provided so that check valve 10c may operate with two different injection fluids such as contrast and saline as examples. Accordingly, single check valve 10c pursuant to this embodiment may be used in place of the dual check valves 10A, 10B associated with the fluid path of syringes 5900A and 5900B of fluid injector system 5000. Check valve 10c operates as a dual check valve and, thereby, may be used in place of check valves 10A, 10B in fluid injector system 5000. Inlet ports 20c(1), 20c(2) may each be formed with a standard threaded female luer connection configuration. However, this specific arrangement should not be considered as exhaustive. One or both of inlet ports 20c(1), 20c(2) could be formed with a standard threaded male luer connection configuration or a combination of a male and female luer connection configuration may be associated with inlet ports 20c(1), 20c(2) as desired. Outlet port 22c may be formed with a standard threaded male or a standard threaded female luer connection configurations as exemplary and non-limiting connecting structures for outlet port 22c.

In this embodiment, housing body 12c does not comprise a single defined internal seal seat within flow passage 18c. More particularly, in this embodiment an internal surface or portion of housing body 12c serves as a seal seat and due to this function will be identified with reference character 24c hereinafter for consistency with previous embodiments. Since two inlet ports 20c(1), 20c(2) are provided in housing body 12c, in practicality two internal seal seats 24c(1), 24c(2) are provided in housing body 12c and defined by an internal surface or portion thereof. Seal seats 24c(1), 24c(2) may general be defined or described as being the opposing internal portions or surfaces of housing body 12c that circumscribe or define opposing internal openings 50c, 52c in housing body 12c which communicate with inlet ports 20c(1), 20c(2). Thus, the interior of housing body 12c in effect defines two seal seats 24c(1), 24c(2) which are respectively associated with inlet ports 20c(1), 20c(2). In view of the foregoing, it will be clear that two respective seal seats 24c(1), 24c(2) are present in flow passage 18c between inlet ports 20c(1), 20c(2) and singular outlet port 22c.

A singular valve member 26c is disposed within the flow passage 18c between inlet ports 20c(1), 20c(2) and outlet port 22c. As in previous embodiments, valve member 26c is positioned and adapted to engage and seal against seal seats 24c(1), 24c(2) and provide a substantially fluid tight seal with each of these elements. In this embodiment, valve member 26c takes a substantially different form from previous embodiments and is in the form of a hollow member 54c that is typically cylindrical or tubular shaped and defines an internal bore or flow passage 56c extending therethrough. In one desirable form, hollow member 54c could be a length of compliant medical tubing that is sized to fit in flow passage 18c in housing body 12c. Such medical tubing is often made of polypropylene for resiliency and compliancy and this is also a suitable material for hollow member 54c. Similarly resilient or compliant materials such rubbers, thermoplastic elastomers, or silicone may be used for hollow member 54c. Hollow member 54c defines a lateral or side opening 58c that is on the lateral side of hollow member 54c facing internal opening 50c and first inlet port 20c(1) which, in the case of an angiographic or computer tomography fluid injection procedure wherein check valve 10c may be used, is typically the saline introduction port to the fluid path leading to the patient.

As with previous embodiments, valve member 26c associated with check valve 10c is generally operable to have at least two flow states including a normally closed position or state wherein the valve member 26c engages seal seats 24c(1), 24c(2). In the closed position or state, valve member 26c engages seal seats 24c(1), 24c(2) but is operable in response to fluid flow in either inlet port 20c(1) or inlet port 20c(2) to move to an open position or state with respect to that port permitting one-directional fluid flow from either inlet port 20c(1) or inlet port 20c(2) to outlet port 22c thereby allowing fluid flow to pass through flow passage 18c from inlet port 20c(1) or inlet port 20c(2) to outlet port 22c. It is noted that the hollow form of valve member 26c makes valve member 26c suitable for use in simultaneous or dual flow situations, wherein two distinct fluids, such as contrast and saline, are simultaneously being injected in a fluid injection procedure. In this situation, opposing sides of hollow member 54c collapse, deflect, or deform inward into internal bore or flow passage 56c thereby allowing fluid from both inlet ports 20c(1), 20c(2) to pass to outlet port 22c. However, in the typical situation wherein sequential fluid injection is occurring, when fluid flow in inlet port 20c(1) or inlet port 20c(2) ceases, valve member 26c is adapted to resiliently return to the normally closed position or state in engagement with either seal seat 24c(1) or seal seat 24c(2). A second or override position or state is specifically provided for valve member 26c to allow bi-directional flow through one of inlet ports 20c(1), 20c(2). In a typical fluid injection procedure involving two fluids such as contrast and saline, the inlet port 20c(1), 20c(2) to be associated with saline is typically the desired port to have the override function or capability as saline is typically used for patency checks. In the override or bypass position, valve member 26c is placed and maintained in the open position or state unseated from seal seat 24c(1) which permits bi-directional fluid flow through the flow passage 18c to outlet port 22c.

In this embodiment, override or bypass actuator 100c is in the form of a plunger override or bypass actuator 100c which is associated with a proximal end 60c of hollow member 54c. Plunger actuator 100c comprises a distal end 110c and a proximal end 112c. A plunger head 114c is provided at the distal end 110c of plunger actuator 100c. A plunger stem 116c extends from plunger head 114c and extends outward from housing body 12c. Plunger head 114c desirably defines a circumferential recess or groove 118c, typically in the form of a circular recess or groove, for engaging the proximal end 60c of hollow member 54c. As hollow member 54c is typically tubular, for example cylindrical, shaped, the proximal end 60c is received and desirably secured in circumferential recess or groove 118c. Any of the conventional joining techniques identified previously may be used to secure this engagement but a medical grade adhesive may be the most convenient way to secure hollow member 54c to plunger head 116c. Alternatively, in this embodiment, plunger actuator 100c may be formed integrally with valve member 26c.

Valve member 26c may be assembled into housing body 12c through an end opening 62c in housing body 12c opposite from outlet port 22c. End opening 62c is enclosed by a cover member 64c that is secured to housing body 12c in end opening 62c by any of the conventional joining techniques identified previously. Plunger stem 116c extends through an opening 66c in cover member 64c. Hollow member 54c forming valve member 26c is desirably sized to fit securely within the internal diameter of housing body 12c but is capable of axial movement in flow passage 18c in response to axial movement (in either direction) of plunger actuator 100c in flow passage 18c. To avoid a situation where the hollow member 54c and plunger actuator 100c are inserted too far axially into flow passage 18c, a stop structure 68c is provided in flow passage 18c and is formed by the internal surface of housing body 12c.

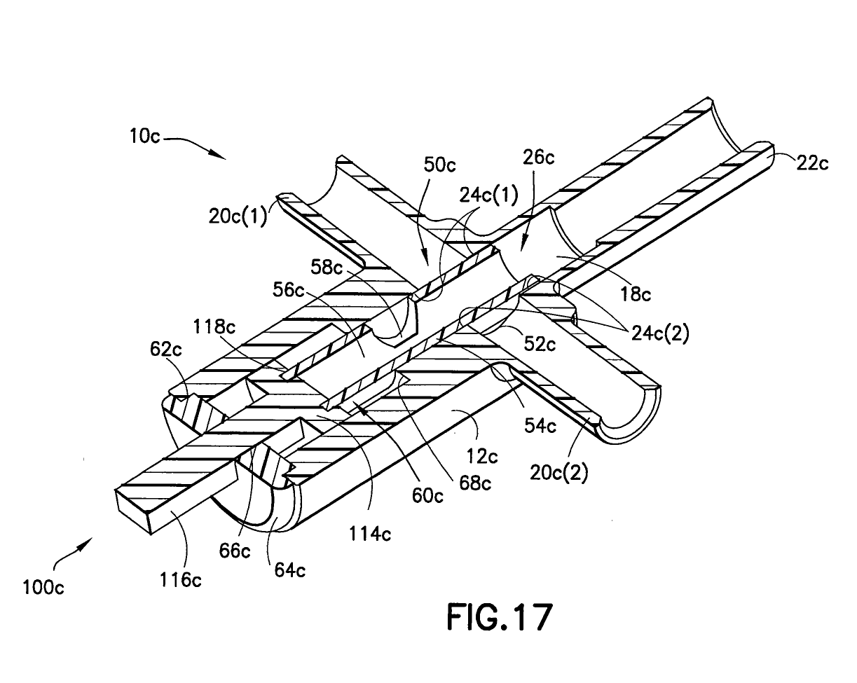

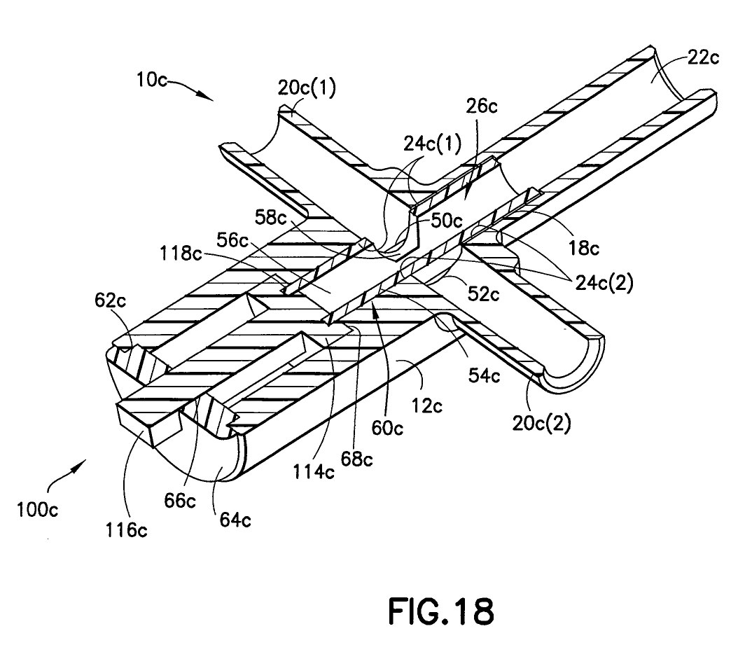

The normally closed position of valve member 26c is shown in FIG. 17 and the override or bypass position of valve member 26c is shown in FIG. 18. In the closed position, as described previously, hollow member 54c forming valve member 26c is seated across seal seats 24c(1), 24c(2) thereby sealing internal openings 50c, 52c. When fluid flow is present in either inlet port 20c(1), 20c(2), the fluid flow acts to deform or compress the hollow member 54c inward into internal bore 56c. This deformation or compression of hollow member 54c causes a gap or opening to form between the hollow member 54c and the internal portion of housing body 12c defining seal seats 24c(1), 24c(2). As result, the respective internal opening 50c, 52c connected to the inlet port 20c(1), 20c(2) experiencing fluid flow is open to permit fluid flow from that port to outlet port 22c. In the simultaneous fluid flow situation described previously, opposing sides of hollow member 54c collapse or deflect or deform inward into internal bore or flow passage 56c thereby creating a gap or opening between the hollow member 54c and seal seats 24c(1), 24c(2) allowing fluid from both inlet ports 20c(1), 20c(2) to pass via internal openings 50c, 52c to outlet port 22c. In the usual closed position of valve member 26c, if a reverse flow situation should occur where fluid flow enters or reverses direction in outlet port 22c, this reverse flow will be channeled into internal bore 56c in hollow member 54c and have the effect of re-sealing hollow member 54c in engagement with opposing seal seats 24c(1), 24c(2) preventing the reverse flow from entering either inlet port 20c(1), 20c(2).

To place valve member 26c in the override or bypass position or state, an operator applies axial pressure to plunger actuator 100c. This applied axial pressure causes axial movement of plunger actuator 100c into housing body 12c and, due to the fixed connection between hollow member 54c forming valve member 26c and the plunger head 114c of plunger actuator 100c, the hollow member 54c moves axially forward or distally in flow passage 18c in housing body 12c. Desirably, stop structure 68c in flow passage 18c is positioned to stop axial movement of plunger head 114c when side opening 58c in hollow member 54c is aligned with internal opening 50c in housing body 12c which communicates or is aligned directly with inlet port 20c(1). As indicated previously, one of inlet ports 20c(1), 20c(2) is often a saline inlet port and since saline is often used for patency check purposes, inlet port 20c(1) is now desirably configured for bi-directional fluid flow for use in conducting patency checks prior to conducting a fluid injection procedure associated with angiographic or computed tomography procedures. Bi-directional fluid flow through flow passage 18c is now enabled through the fluid communication between inlet port 20c(1) and outlet port 22c. In particular, with side opening 58c aligned with internal opening 50c, bidirectional fluid communication is established between inlet port 20c(1) and outlet port 22c. This fluid path extends from inlet port 20c(1) to outlet port 22c via internal opening 50c in housing body 12c, side opening 58c in hollow member 54c, and internal bore 56c in hollow member 54c which is aligned coaxially with flow passage 18c leading to outlet port 22c. It will be clear that any fluid flow passing through internal bore 56c in hollow member 54c has the effect of securing the seated engagement of hollow member 54c against the opposing seal seat 24c(2) associated with opposing internal opening 52c. However, even in this situation, it may be possible to introduce fluid flow in inlet port 20c(2) that will deform hollow member 54c sufficiently to allow fluid flow to pass from inlet port 20c(2) to outlet port 22c while valve member 26c is in the override or bypass position, such as may occur in a simultaneous or dual flow situation.

Due to the axially movable engagement of hollow member 54c in flow passage 18c, if reverse pressurized fluid flow is encountered in flow passage 18c as, for example, if reverse pressurized fluid flow occurs in outlet port 22c, hollow member 54c will automatically reset to its initial or closed position. In particular, in a reverse pressurized fluid flow situation, the reverse fluid flow enters central bore 56c in hollow member 54c and acts against plunger head 114c provided at the distal end 110c of plunger actuator 100c. The reverse or proximally directed force generated by the reverse fluid flow causes the plunger actuator 100c to move proximally in flow passage 18c, thereby also moving hollow member 54c proximally in flow passage 18c due to the generally fixed connection between plunger head 114c and the proximal end 60c of the hollow member 54c. In this way, valve member 26c formed by hollow member 54c in this embodiment is reset to its initial, closed position and, accordingly, valve member 26c comprises an automatic reset function in this embodiment.

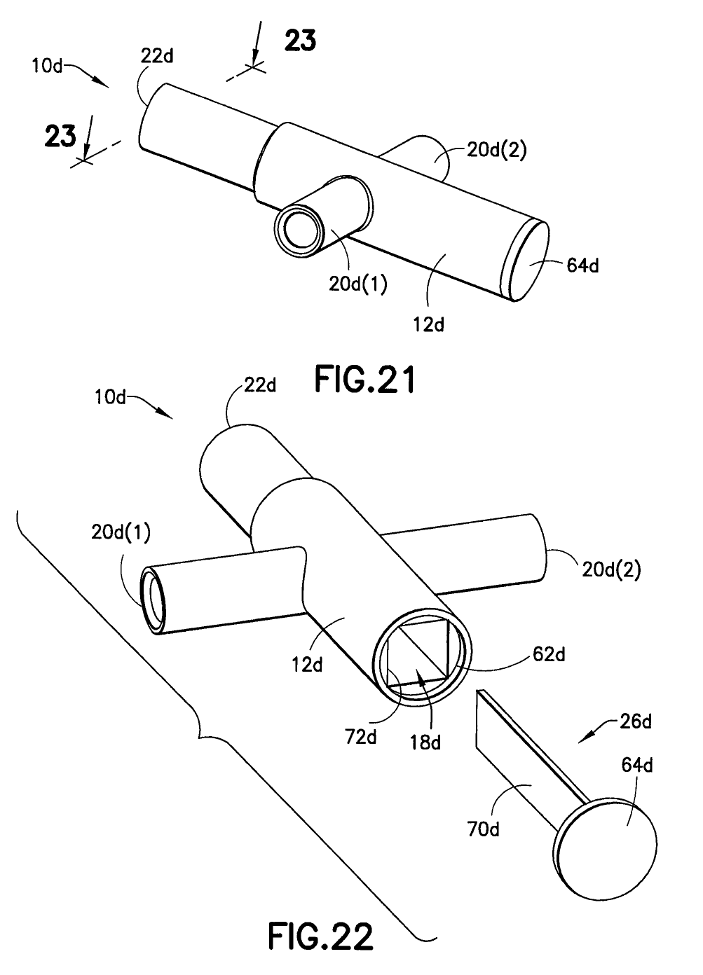

A fourth embodiment of check valve 10d is shown in FIGS. 21-24. Check valve 10d according to this embodiment comprises a housing body 12d which is substantially identical to the housing body 12c of check valve 10c and, thus, the details of housing body 10d are not recited hereinafter. In this embodiment, valve member 26d has a substantially different form and operation from valve member 26c discussed immediately above. Accordingly, the form and operation of valve member 26d serve as the main differences in check valve 10d in comparison to check valve 10c discussed previously. Valve member 26d in this embodiment comprises a cantilever valve member 70d which is typically formed integral with cover member 64d used to enclose end opening 62d in housing body 12d. Cover member 64d may be secured in end opening 62d by any of the conventional joining techniques identified previously. As an alternative cantilever valve member 70d may be formed separately from cover member 64d and secured to cover member 64d, again by any of the conventional joining techniques identified previously. End opening 62d includes a polygonal shaped area 72d in the shape of a square in the illustrated embodiment that is adapted to receive a corresponding polygonal shaped portion 74d formed on cover member 64d. Such a polygonal-polygonal mating engagement prevents cover member 64d from rotating relative to housing body 12d during assembly and an additional advantage of this mating engagement is the proper positioning of cantilever valve member 70d generally along a centerline or central axis of CL flow passage 18d.

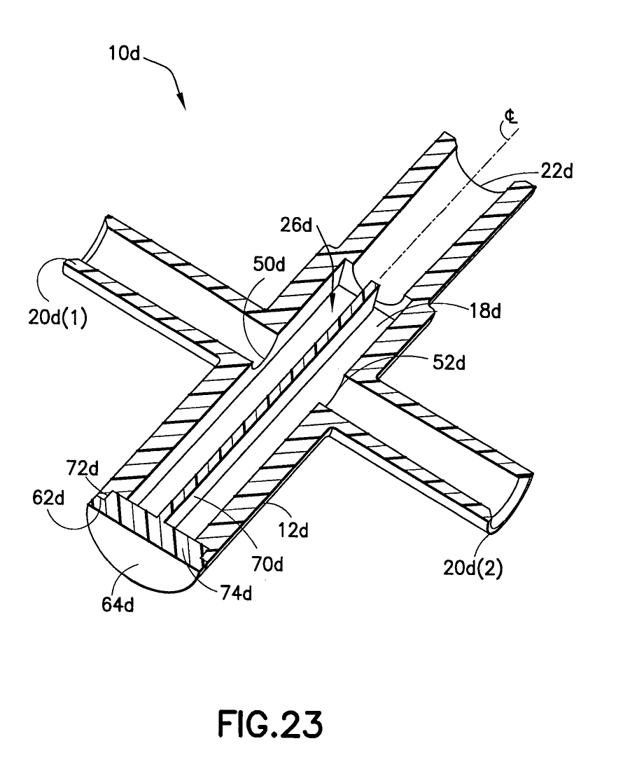

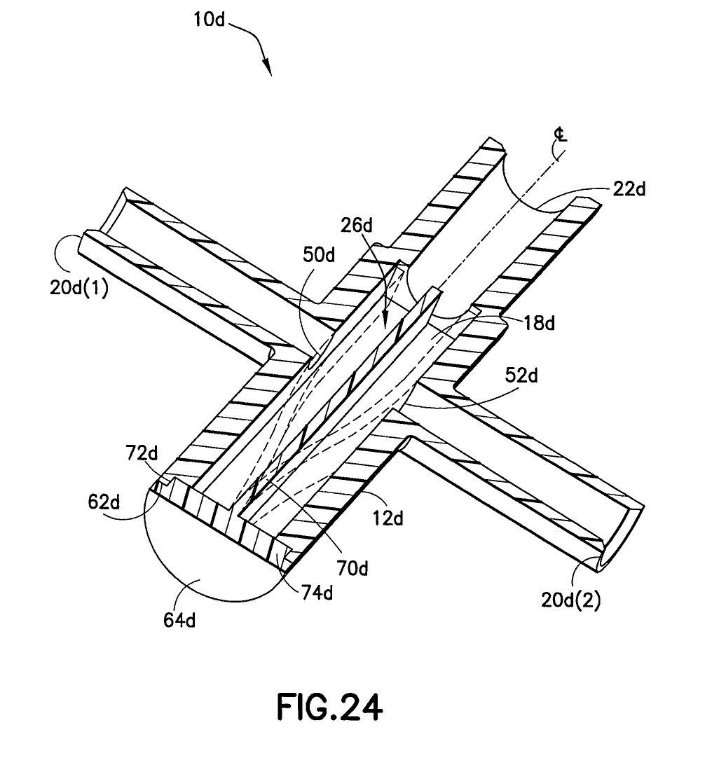

As illustrated in FIGS. 23-24, flow passage 18d is formed to accommodate cantilever valve member 70d and side-to-side movement thereof in flow passage 18d in response to fluid flow in flow passage 18d as described herein. This side-to-side movement is in response to fluid flow from either inlet port 20c(1) or inlet port 20c(2) or both. Cantilever valve member 70d is desirably a resilient leaf spring structure that adjusts according to fluid flow conditions in flow passage 18d. In contrast to previous embodiments, cantilever valve member 70d is normally in the position illustrated in FIG. 23 and generally positioned along central axis CL flow passage 18d and, thus, does not block fluid flow through either lateral internal opening 50d, 52d in housing body 12d in the normal position or state. Accordingly, the normal position or state of cantilever valve member 70d is an open position or state wherein the cantilever valve member 70d does not seat against either of laterally disposed seal seats 24d(1), 24d(2) in housing body 12d. Cantilever valve member 70d only seats against or engages one of seal seats 24d(1), 24d(2) when fluid flow is present in either inlet port 20d(1) or inlet port 20d(2), or possibly both ports. As will be clear from the foregoing, cantilever valve member 70d is self-adjusting to fluid flow in flow passage 18d and there is no ability to override the functioning of cantilever valve member 70d as in previous embodiments. However, due to the normally open position or state of cantilever valve member 70d patency checks may be accomplished via either inlet port 20d(1), 20d(2), or possibly via both ports.

In the normal operation of check valve 10d wherein fluid flow is present one of inlet ports 20d(1), 20d(2), for example, inlet port 20d(1), fluid flow in inlet port 20d(1) passes unobstructed through internal opening 50d and causes or forces cantilever valve member 70d to move toward the unpressurized internal opening 52d and seal seat 24d(2) until the valve member 70d engages seal seat 24d(2) and seals opposing internal opening 52d. Fluid flow from inlet port 20d(1) is able to pass without restriction to outlet port 22d. Valve member 26d operates in a similar manner to the foregoing if fluid flow is present in inlet port 20d(2) only. If simultaneous flow is present in inlet ports 20c(1), 20c(2) cantilever valve member 70d adjusts accordingly. A simultaneous fluid injection situation wherein fluid flow is present in both inlet ports 20d(1), 20d(2) could occur when it is desired to inject, for example, saline and contrast during an angiographic or computed tomography procedure. Cantilever valve member 70d adjusts in flow passage 18d according to the relative fluid pressure between inlet ports 20d(1), 20d(2) acting on the cantilever valve member 70d. If one side of cantilever valve member 70d is under greater pressure than the other side, the cantilever valve member 70d adjust to the low pressure side and may in part or in total block fluid flow from the lower pressure inlet port, typically inlet port 20d(1) in a simultaneous saline-contrast fluid injection situation. If fluid pressure in inlet ports 20d(1), 20d(2) are somewhat equal cantilever valve member 70d may have the substantially centerline orientation of FIG. 23.

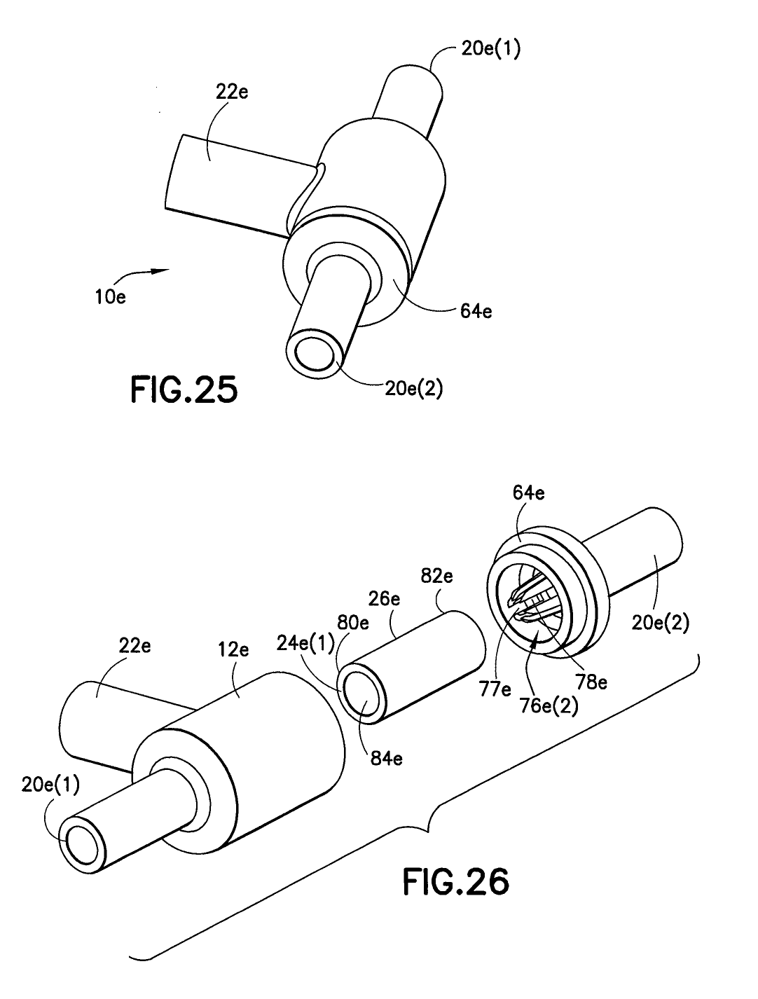

A fifth embodiment of check valve 10e is shown in FIGS. 25-29. Check valve 10e according to this embodiment typically comprises a unitary housing body 12e which defines an internal flow passage 18e for fluid flow through the housing body 12e. In this embodiment, housing body 12e defines a pair of opposing first and second inlet port 20e(1), 20e(2) and an outlet port 22e which communicate with flow passage 18e in a similar manner to several of the foregoing embodiments. Dual inlet ports 20e(1), 20e(2) are again provided so that check valve 10e may operate with two different injection fluids such as contrast and saline as examples. Accordingly, single check valve 10e pursuant to this embodiment may also be used in place of dual check valves 10A, 10B associated with the fluid path of syringes 5900A and 5900B of fluid injector system 5000. Check valve 10e operates as a dual check valve and, thereby, may be used in place of check valves 10A, 10B in fluid injector system 5000 in a substantially similar manner to check valve 10c discussed previously. As in previous embodiments, inlet ports 20e(1), 20e(2) may each be formed with a standard threaded female luer connection configuration. However, this specific arrangement should not be considered as definitive. One or both of inlet ports could be formed with a standard threaded male luer connection configuration or a combination of a male and female luer connection configuration may be associated with inlet ports 20e(1), 20e(2) as desired. Outlet port 22e may be formed with a standard threaded male luer connection configuration or a standard threaded female luer connection configuration as exemplary and non-limiting connecting structures for outlet port 22e.

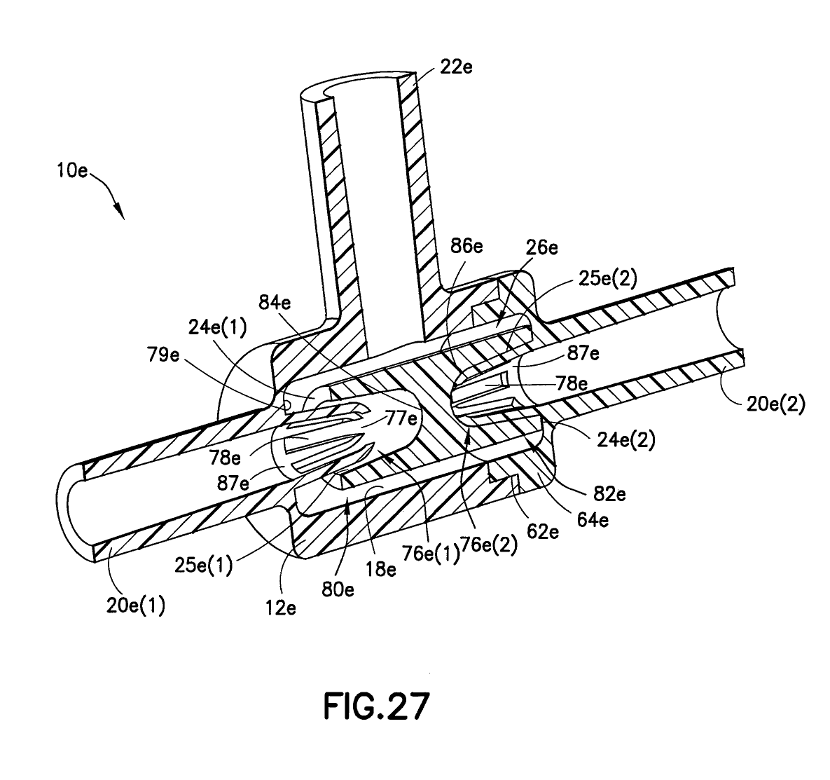

Inlet ports 20e(1), 20e(2) each comprising an inlet port member 76e(1), 76e(2), respectively, extending into flow passage 18e from opposing sides of flow passage 18e. The respective port members 76e(1), 76e(2), or first and second inlet port members 76e(1), 76e(2), may be slotted dome structures which define a plurality of slots or openings for fluid passage laterally outward from first and second inlet port members 76e(1), 76e(2). More particularly, first and second port inlet members 76e(1), 76e(2) define distal exit openings 77e and are generally segmented with slots 78e which permits fluid flow to exit laterally from the first and second inlet port members 76e(1), 76e(2) as well axially along a central axis of the first and second inlet port members 76e(1), 76e(2) via distal exit openings 77e. A proximal end portion 79e of each of the first and second inlet port members 76e(1), 76e(2) is formed as an annular end structure that is adapted to form a fluid seal with seal seats 24e(1), 24e(2) associated with valve member 26e in this embodiment as discussed herein. Valve member 26e is disposed in flow passage 18e and comprises opposing ends 80e, 82e which are formed for association or cooperating engagement with first and second inlet port members 76e(1), 76e(2). Valve member 26e is generally cylindrical shaped and defines recesses 84e, 86e in opposing ends 82e, 84e thereof which are adapted to receive the opposing first and second inlet port members 76e(1), 76e(2). Seal seats 24e(1), 24e(2), in this embodiment, are defined at the opposing ends 80e, 82e for sealing against the proximal annular end portion 79e associated with the first and second inlet port members 76e(1), 76e(2), respectively, to regulate fluid flow through flow passage 18e. As shown in cross section in FIG. 27, for example, inlet port 20e(2) may form a cover member 64e in this embodiment closing end opening 62e in housing body 12e which is typically used to assemble valve member 26e into flow passage 18e. Accordingly, inlet port 20e(2) may be secured in end opening 62e by any of the conventional joining techniques identified previously.

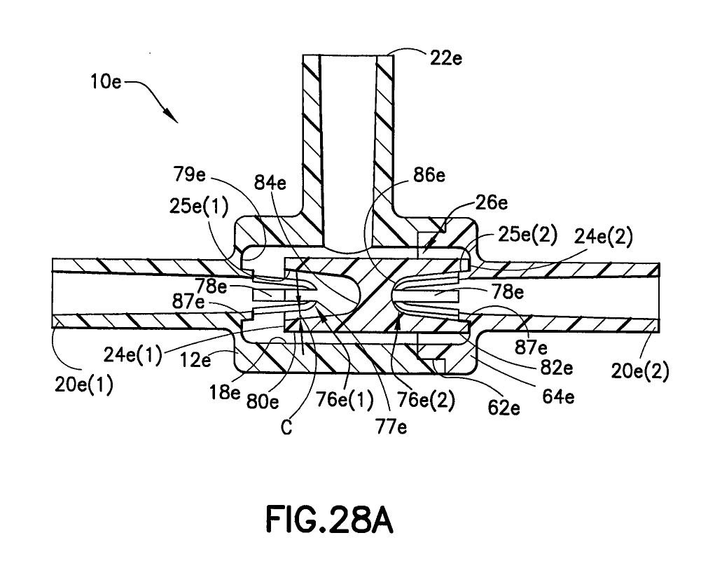

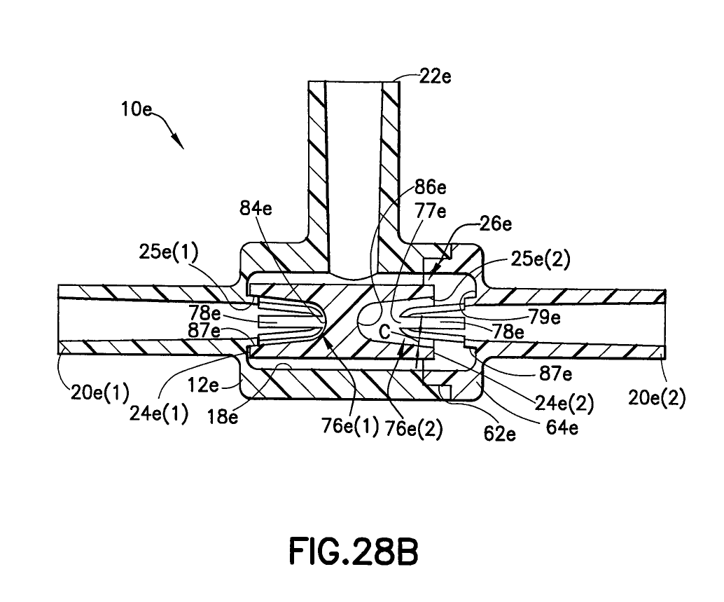

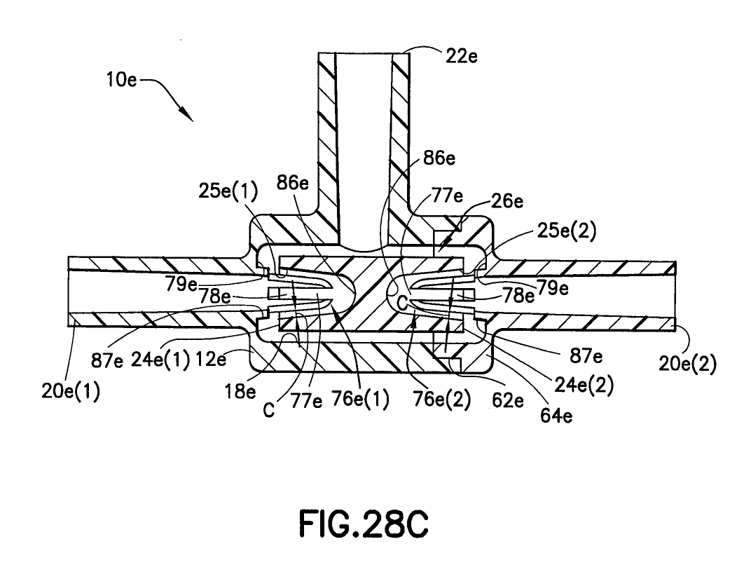

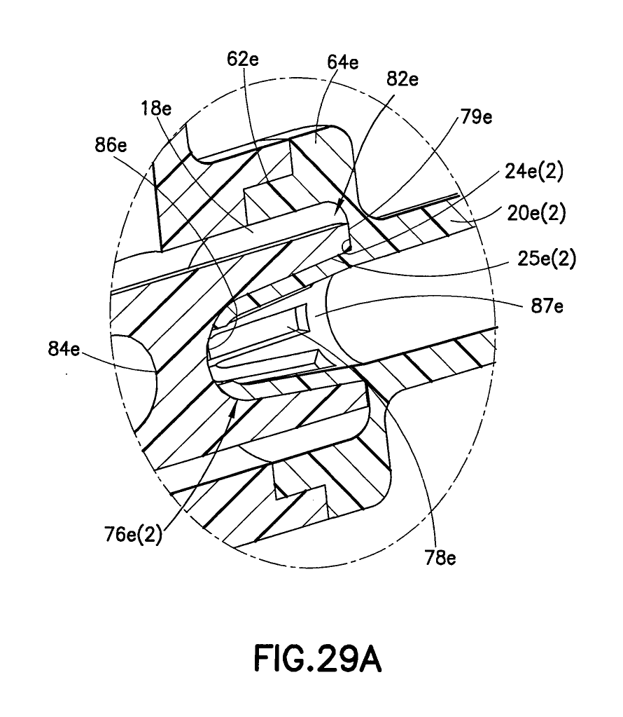

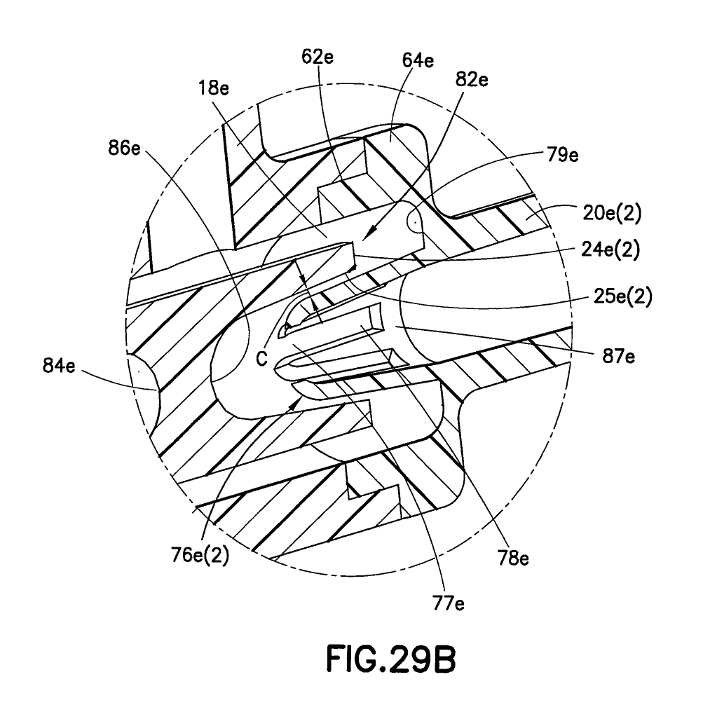

It will be clear from the foregoing described structure that valve member 26e operates as a shuttlecock valve member 26e and is self-adjusting to fluid flow in flow passage 18e in a similar manner to valve member 26c discussed previously in connection check valve 10c. Accordingly, there is again no ability based on the structure of shuttlecock valve member 26e and first and second inlet port members 76e(1), 76e(2) to physically override the functioning of shuttlecock valve member 26e. Instead, valve member 26e is fluid flow responsive to fluid flow in one or both of first and second inlet ports 20e(1), 20e(2) to form multiple states as described herein. In the normal operation of check valve 10e wherein fluid flow is present one of inlet ports 20e(1), 20e(2), for example, inlet port 20e(1), fluid flow in inlet port 20e(1) passes through first inlet port member 76e(1) and laterally outward through slots 78e in first inlet port member 76e(1) as well axially outward from distal exit opening 77e defined by the first inlet port member 76e(1). If first inlet port member 76e(1) is initially sealed with its proximal end portion 79e in engagement with seal seat 24e(1) thereby placing inlet port 20e(1) in a closed state, fluid pressure in inlet port 20e(1) exerts a pressure force in recess 84e and, thereby, on shuttlecock valve member 26e causing shuttlecock valve member 26e to move laterally toward the opposing second inlet port member 76e(2) and, accordingly, axially within flow passage 18e. As shuttlecock valve member 26e moves toward second inlet port member 76e(2), the proximal end portion 79e of second inlet port member 76e(2) engages seal seat 24e(2) defined at the second end 82e of shuttlecock valve member 26e. This seals opposing inlet port 20e(2) from fluid communication with flow passage 18e, as illustrated in FIG. 28A and in detail in FIG. 29A. However, simultaneously, fluid communication is established between first inlet port 20e(1) and flow passage 18e via spacing or clearance C that is formed between first inlet port member 76e(1) and end recess 84e as shuttlecock valve member 26e moves laterally away from inlet port 20e(1) and toward opposing inlet port 20e(2). Accordingly, fluid flow is able to exit first inlet port member 76e(1) and pass to outlet port 22d via end recess 84e and flow passage 18e. Shuttlecock valve member 26e operates in a generally reverse manner to the foregoing if fluid flow is present in second inlet port 20e(2) only and moves to the position shown in FIG. 28B. A detail view of shuttlecock valve member 26e when moved laterally away from second inlet port member 76e(2) permitting fluid flow to pass from second inlet port 20e(2) to flow passage 18e is shown FIG. 29B. It will be noted that secondary seal seats 25e(1), 25e(2) are formed just within end recesses 84e, 86e, respectively. Secondary seal seats 25e(1), 25e(2) are, in particular, the inner peripheral edge or surface of end recesses 84e, 86e that receives and engages the outer surface of an annular band portion 87e associated with each inlet port member 76e(1), 76e(2). The engagement of the outer surface of annular band portion 87e associated with each inlet port member 76e(1), 76e(2) and the respective seal seats 25e(1), 25e(2) enhances the fluid sealing characteristics of valve member 26e in this embodiment by providing an additional sealing surface engagement between valve member 26e and the respective inlet port members 76e(1), 76e(2) to compliment or supplement the sealing engagement provided by the seal seats 24e(1), 24e(2) associated with valve member 26e engaging the proximal end portions 79e of inlet port members 76e(1), 76e(2). It will be further noted that when one side of shuttlecock valve member 26e is under fluid pressure thereby causing the valve member 26e to form a generally fluid tight seal with the opposing inlet port member 76e(1) or 76e(2) in the manner described hereinabove this generally fluid tight seal or engagement increases with increasing fluid pressure. In other words, as fluid pressure increases at one end of valve member 26e, the robustness of the opposing sealing engagement increases at the other end.

If simultaneous flow is present in inlet ports 20e(1), 20e(2) shuttlecock valve member 26e adjusts accordingly. A simultaneous fluid injection situation wherein fluid flow is present in both inlet ports 20e(1), 20e(2) could occur, as discussed previously, when it is desired to inject, for example, saline and contrast during an angiographic or computed tomography (“CT”) procedure. Shuttlecock valve member 26e adjusts laterally in flow passage 18e according to the relative fluid pressure between inlet ports 20e(1), 20e(2) acting on the shuttlecock valve member 26e. If higher fluid pressure is present in one of inlet ports 20e(1), 20e(2), shuttlecock valve member 26e adjusts in position toward the lower pressure port and potentially may seal the lower pressure inlet port, typically inlet port 20e(1) in a simultaneous saline-contrast fluid injection situation, by engagement of the proximal end portion 79e of first inlet port member 76e(1) with “first” seal seat 24e(1) associated with end 80e of valve member 26e. If fluid pressure in inlet ports 20e(1), 20e(2) is somewhat equal shuttlecock valve member 26e may have a substantially centered axial orientation in flow passage 18e as illustrated in FIG. 28C thereby allowing fluid communication between both inlet ports 20e(1), 20e(2) and outlet port 22e. In view of the foregoing, valve member 26e may exhibit a first state wherein fluid communication is established between first inlet port 20e(1) and outlet port 22e while fluid communication is prevented between second inlet port 20e(2) and outlet port 22e; a second state wherein fluid communication is established between second inlet port 20e(2) and outlet port 22e while fluid communication is prevented between first inlet port 20e(1) and outlet port 22e; and a third state wherein fluid communication is at least partially present between both inlet ports 20e(1), 20e(2) and outlet port 22e. With valve member 26e in either the first state or the second state, a patency check may be conducted with the open inlet, namely first inlet port 20e(1) or second inlet port 20e(2). It is also noted that a patency check may be conducted with either open inlet port in the third state as both the first and second inlet ports 20e(1), 20e(2) are at least partially open for bi-directional fluid flow.

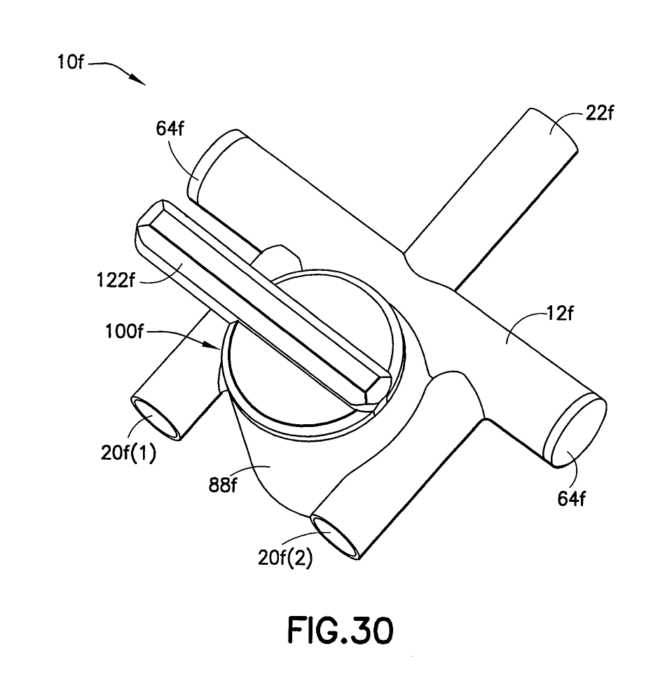

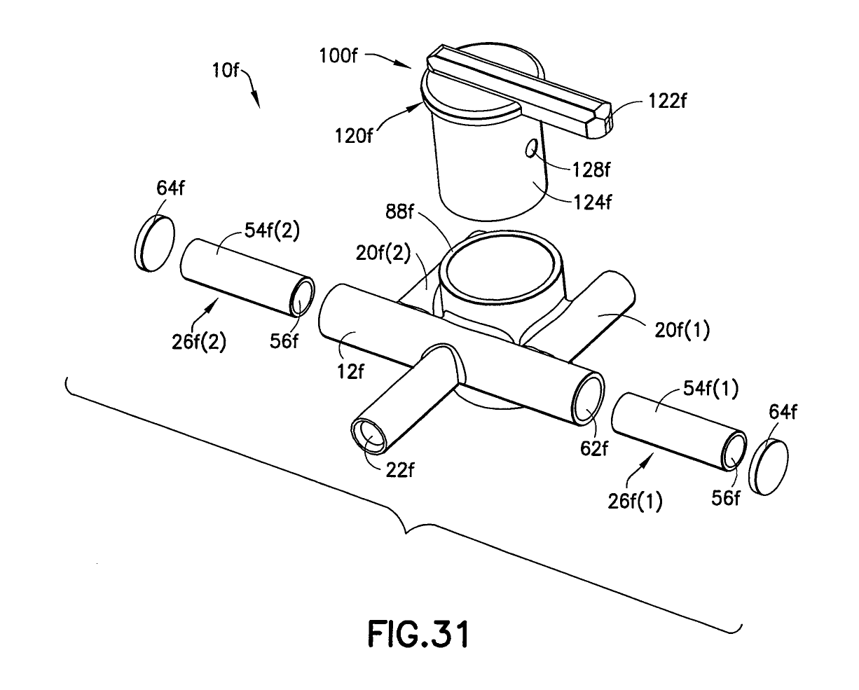

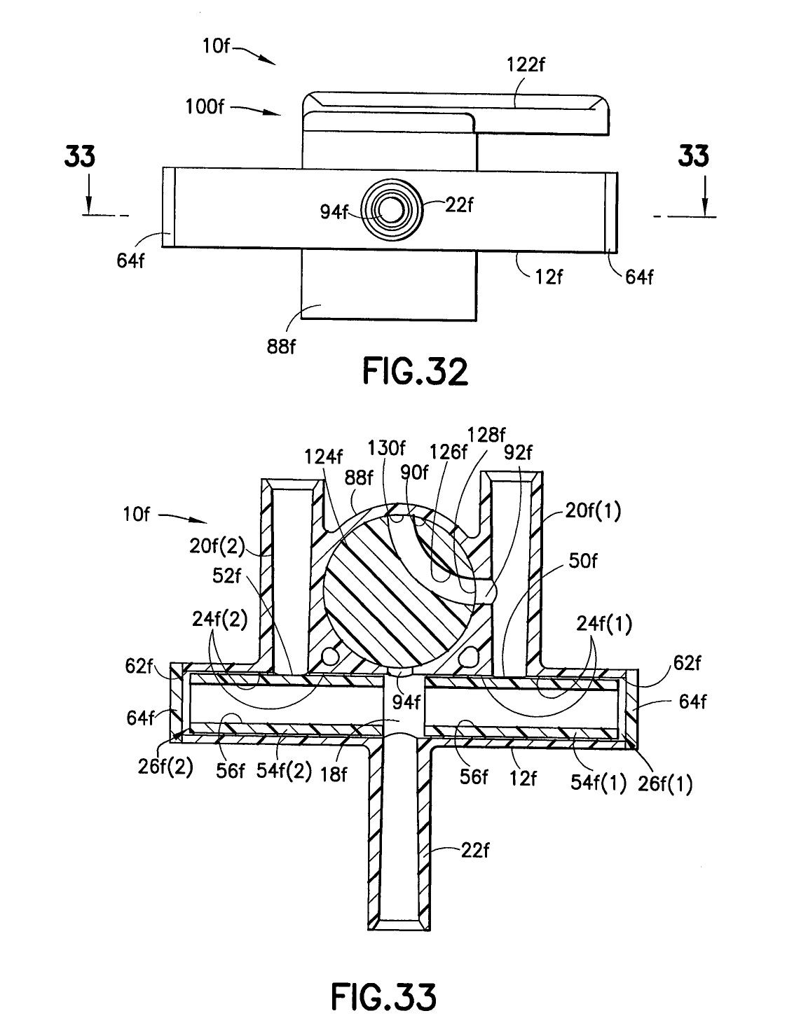

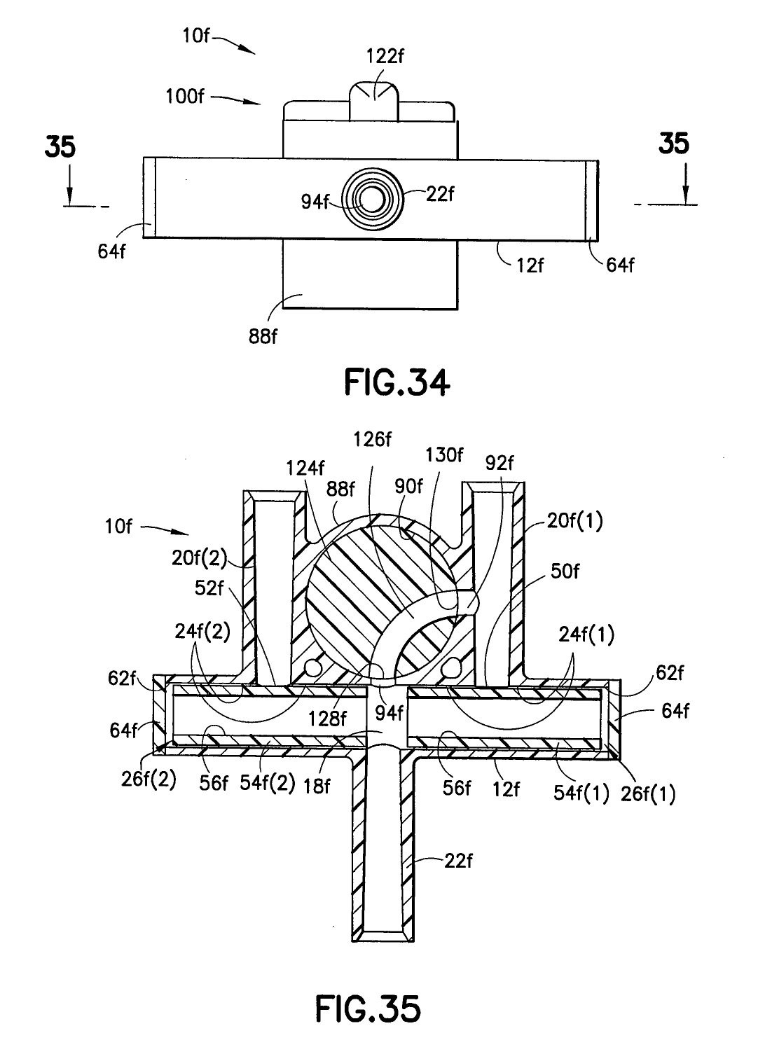

A sixth embodiment of check valve 10f is shown in FIGS. 30-35. Check valve 10f according to this embodiment has certain similarities to check valve 10c discussed previously. Accordingly, the following discussion draws from certain aspects of check valve 10c discussed previously. As with this previous embodiments, check valve 10f comprises a unitary housing body 12f which defines an internal flow passage 18f for fluid flow through the housing body 12f. In this embodiment, housing body 12f again defines a pair of opposing first and second inlet port 20f(1), 20f(2) and an outlet port 22f which communicate with flow passage 18f. Inlet ports 20f(1), 20f(2) in contrast to check valve 10c are oriented generally parallel with outlet port 22f rather the generally perpendicular orientation of inlet ports 20c(1), 20c(2) in check valve 10c. Inlet ports 20f(1), 20f(2) are again provided so that check valve 10f may operate with two different injection fluids such as contrast and saline as examples, and check valve 10f may be used in place of dual check valves 10A, 10B associated with the fluid path of syringes 5900A and 5900B of fluid injector system 5000. Check valve 10f operates as a dual check valve and, thereby, may be used in place of check valves 10A, 10B in fluid injector system 5000. Inlet ports 20f(1), 20f(2) may each be formed with a standard threaded female luer connection configuration. However, this specific arrangement should not be considered as definitive. One or both of inlet ports could be formed with a standard threaded male luer connection configuration or a combination of a male and female luer connection configuration may be associated with inlet ports 20f(1), 20f(2) as desired. Outlet port 22f may be formed with a standard threaded male connection configuration or a standard threaded female connection configuration as exemplary and non-limiting connecting structures for outlet port 22f.

In this embodiment, housing body 12f does not comprise a single defined internal seal seat within flow passage 18f. More particularly, in this embodiment an internal surface or portion of housing body 12f serves as a seal seat and due to this function will again be identified with reference character 24f hereinafter for consistency with previous embodiments, particularly check valve 20c. Since two inlet ports 20f(1), 20f(2) are provided in housing body 12f, in practicality two internal seal seats 24f(1), 24f(2) are provided in housing body 12f and defined by an internal surface or portion thereof. Seal seats 24f(1), 24f(2) may generally be defined or described as being the opposing internal portions or surfaces of housing body 12f that circumscribe or define internal openings 50f, 52f in housing body 12f which communicate with inlet ports 20f(1), 20f(2). Thus, the interior of housing body 12f in effect defines two seal seats 24f(1), 24f(2) which are respectively associated with inlet ports 20f(1), 20f(2). In view of the foregoing, it will be clear that two respective seal seats 24f(1), 24f(2) are present in flow passage 18f between inlet ports 20f(1), 20f(2) and singular outlet port 22f.

In contrast to check valve 10c, a pair of valve members 26f(1), 26f(2), comprising a first valve member 26f(1) and a second valve member 26f(2), is disposed within the flow passage 18f and are associated with inlet ports 20c(1), 20c(2), respectively. Outlet port 22f is in fluid communication with flow passage 18f as illustrated, for example, in FIG. 33. Valve members 26f(1), 26f(2) are positioned and adapted to engage and seal against seal seats 24c(1), 24c(2), respectively, and provide a substantially fluid tight seal with each of these elements to control fluid flow through internal openings 50f, 52f in housing body 12f. In this embodiment, valve members 26f(1), 26f(2) take the form of opposing hollow members 54f(1), 54f(2) which are again tubular and typically cylindrical shaped and each define an internal bore or flow passage 56f extending therethrough in fluid communication with flow passage 18f. In one desirable form, hollow members 54f(1), 54f(2) could be a length of compliant medical tubing that is sized to fit in flow passage 18f in housing body 12f. Such medical tubing is often made of polypropylene for resiliency and compliancy and this is also a suitable material for hollow member 54f(1), 54f(2). Similarly resilient or compliant materials such rubbers, thermoplastic elastomers, or silicone may be used for hollow members 54f(1), 54f(2).

As with previous embodiments, valve member 26f(1), 26f(2) associated with check valve 10f are generally operable to have at least two flow states including a normally closed position or state wherein the respective valve members 26f(1), 26f(2) engage seal seats 24f(1), 24f(2). In the closed position or state, valve members 26f(1), 26f(2) engage seal seats 24f(1), 24f(2), respectively, but are operable in response to fluid flow in either inlet port 20f(1) or inlet port 20f(2) (or both in a simultaneous fluid flow situation) to move to an open position or state with respect to that port permitting one-directional fluid flow from either inlet port 20f(1) or inlet port 20f(2) (or both in a simultaneous fluid flow situation) to outlet port 22f thereby allowing fluid flow to pass through flow passage 18f from inlet port 20f(1) or inlet port 20f(2) (or both in a simultaneous fluid flow situation) to the outlet port 22f. As the foregoing discussion makes clear, the hollow and deformable form of valve members 26f(1), 26f(2) makes valve members 26f(1), 26f(2) suitable for use in simultaneous or dual flow situations, wherein two distinct fluids, such as contrast and saline, are simultaneously being injected in a fluid injection procedure. In this situation, hollow members 54f(1), 54f(2) collapse or deflect or deform inward into their internal bores 56f and unseat from their respective engagements with seal seats 24f(1), 24f(2) sufficiently to allow fluid from both inlet ports 20f(1), 20f(2) to pass to outlet port 22f. However, in the typical situation wherein sequential fluid injection is occurring, when fluid flow in either inlet port 20f(1) or inlet port 20f(2) ceases, the deformed valve member 26f(1), 26f(2) is adapted to resiliently return to the normally closed position or state in engagement with either seal seat 24f(1) or seal seat 24f(2). An override or bypass state or position is now specifically provided for valve member 26f(1) to allow bi-directional flow through inlet port 20f(1) in this embodiment. As described previously, in a typical fluid injection procedure involving two fluids such as contrast and saline, one of inlet ports 20f(1), 20f(2), inlet port 20f(1) in the present example, is associated with saline and check valve 10f desirably has an override function or capability with respect to valve member 26f(1) for patency check purposes. In the override or bypass position or state, valve member 26f(1) is adapted to be entirely bypassed which permits bidirectional fluid flow between inlet port 20f(1) and outlet port 22f through flow passage 18f.

In this embodiment, override or bypass actuator 100f is in the form of a bypass cylinder lever actuator 100f which is rotatably associated with a cylindrical housing portion 88f defined by housing body 12f. Cylindrical housing portion 88f is typically formed integral with housing body 12f and is disposed between inlet ports 20f(1), 20f(2). Cylindrical housing portion 88f defines a cylindrical cavity or recess 90f adapted to receive cylinder lever actuator 10f. As illustrated in FIGS. 33 and 35, cylindrical housing portion 88f defines a side port 92f communicating with inlet port 20f(1) and an interface port 94f communicating with flow passage 18f. Cylinder lever actuator 100f comprises a top end 120f with a lever member 122f for actuating the cylinder lever actuator 100f and a depending cylindrical portion 124f adapted for reception and rotatable securement in cylindrical cavity or recess 90f defined by cylindrical housing portion 88f. Cylindrical portion 124f defines a bypass passage 126f of generally curved or arcuate shape therethrough, typically in one quadrant thereof. Cylindrical lever actuator 100f is seated for rotational movement in cylindrical recess or cavity 90f between at least a first position as shown in FIG. 33 wherein bypass passage 126f is in fluid communication at a first end 128f with side port 92f but is blocked at a second end 130f by the internal sidewall 96f of cylindrical housing portion 88f defining cylindrical cavity/recess 90f, and a second position wherein the first end 128f is rotated to a position in fluid communication with flow passage 18f and the second end 130f is in fluid communication with side port 92f thereby allowing bypass passage 126f to provide two-way fluid communication between inlet port 20f(1) an outlet port 22f.

Valve members 26f(1), 26f(2) may be assembled into housing body 12f through opposing end openings 62f(1), 62f(2) in housing body 12f. End openings 62f(1), 62f(2) are enclosed by respective cover members 64f(1), 64f(2) which are secured to housing body 12f in end openings 62f(1), 62f(2) by any of the conventional joining techniques identified previously in this disclosure. Valve members 26f(1), 26f(2) may be constrained from axial movement in flow passage 18f by mechanical stop engagement in housing body 12f or by appropriately placed adhesive securement between valve members 26f(1), 26f(2) and the inner surface of housing body 12f. Further, cylinder lever actuator 100f desirably forms a generally fluid tight seal with housing body 12f when assembled therewith but remains rotatable relative to housing body 12f.

The normally closed position or state of valve members 26f(1), 26f(2) is shown in FIG. 33 and the override or bypass position of valve member 26f(1) is shown in FIG. 35. In the closed position, as described previously, hollow members 54f(1), 54f(2) forming valve members 26f(1), 26f(2) are seated across seal seats 24f(1), 24f(2) thereby sealing internal openings 50f, 52f. When fluid flow is present in either inlet port 20f(1), 20f(2), the fluid flow acts to deform or compress the respective hollow member 54f(1), 54f(2) encountering fluid flow inward into its internal bore or flow passage 56f. This deformation or compression of the respective hollow members 54f(1), 54f(2) causes a gap or opening to form between the hollow member 54f(1), 54f(2) and the internal portion of housing body 12f defining seal seats 24f(1), 24f(2). As a result, the respective internal opening 50f, 52f connected to the inlet port 20f(1), 20f(2) experiencing fluid flow is open to permit fluid flow from that port to outlet port 22f. In the simultaneous or dual fluid flow situation described previously, both hollow members 54f(1), 54f(2) collapse or deflect or deform inward into their internal bores or flow passages 56f thereby creating a gap or opening between the respective hollow members 54f(1), 54f(2) and seal seats 24f(1), 24f (2) allowing fluid from both inlet ports 20f(1), 20f(2) to pass via internal openings 50f, 52f to outlet port 22f. In the usual closed position of valve members 26f(1), 26f(2), if a reverse fluid flow situation should occur where fluid flow enters or reverses direction in outlet port 22f, this reverse flow will be channeled into the internal bores 56f in hollow members 54f(1), 54f(2) and have the effect of expanding and sealing hollow members 54f(1), 54f(2) in engagement with its opposing seal seat 24f(1), 24f(2) preventing such reverse flow from entering either inlet port 20f(1), 20f(2).

To place valve member 26f(1) in the override or bypass position or state, an operator rotates cylinder lever actuator 100f 90° counter clockwise from the orientation shown in FIG. 33 to the orientation shown in FIG. 35. This movement causes cylindrical portion 124f to rotate from the first position as shown in FIG. 33, wherein bypass passage 126f is in fluid communication at first end 128f with side port 92f but is blocked at second end 130f by the internal sidewall 96f of cylindrical housing portion 88f, to the second or bypass position shown in FIG. 35. In this second or bypass position, the first end 128f of bypass passage 126f is in fluid communication with flow passage 18f and the second end 130f of bypass passage 126f is in fluid communication with side port 92f. In this second or bypass position, bypass passage 126f provides two-way fluid communication between inlet port 20f(1) an outlet port 22f. As indicated previously, one of inlet ports 20f(1), 20f(2) is often a saline inlet port and since saline is often used for patency check purposes, inlet port 20f(1) is now desirably configured for bi-directional fluid flow for use in conducting patency checks prior to conducting a fluid injection procedure associated with angiographic or computed tomography procedures. Bi-directional fluid flow through flow passage 18f is now enabled through the fluid communication between inlet port 20f(1) and outlet port 22f provided by bypass passage 126f.

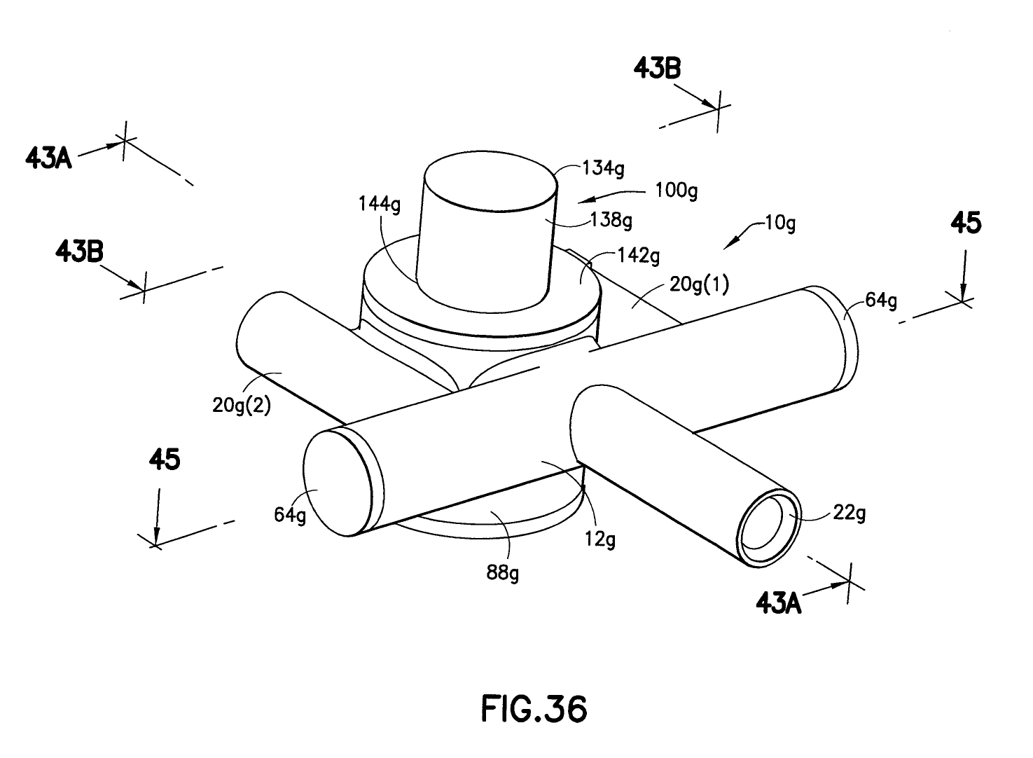

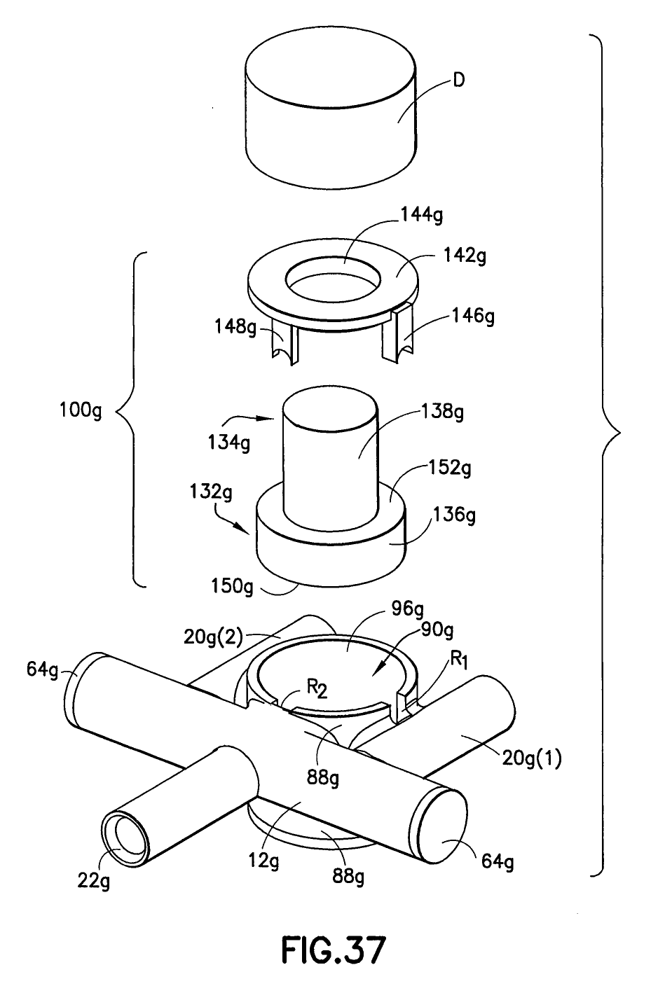

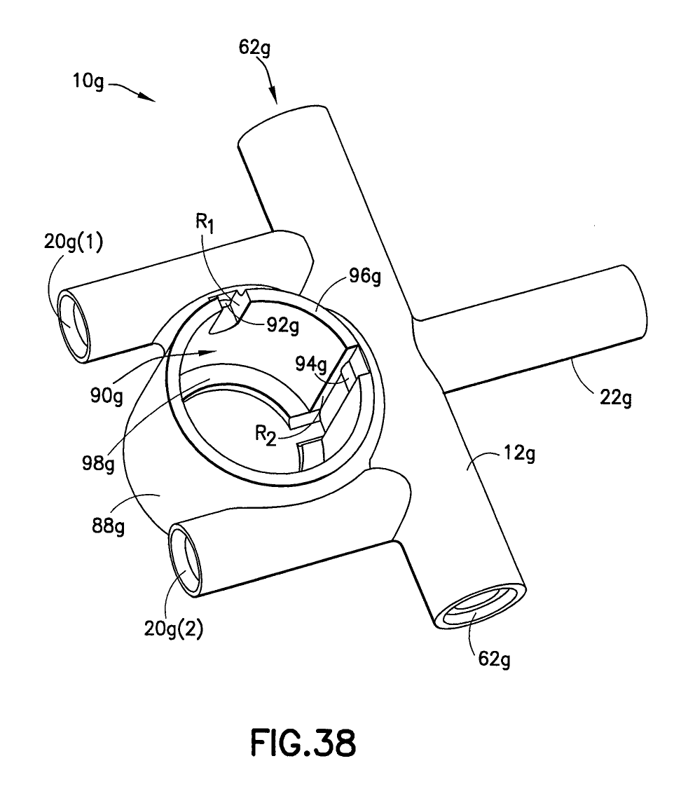

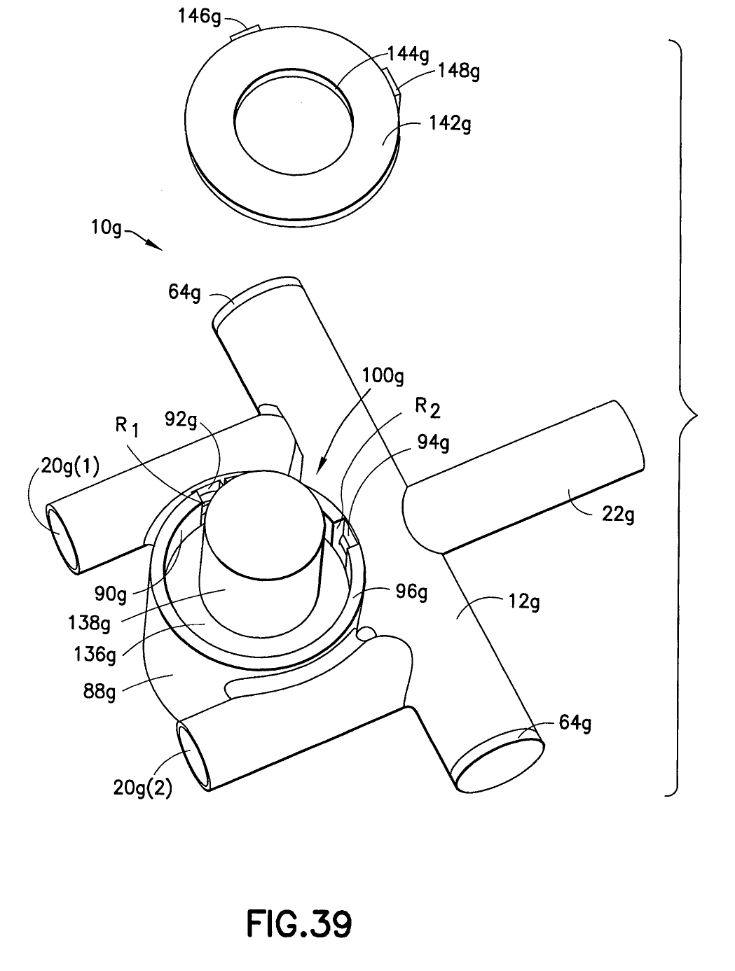

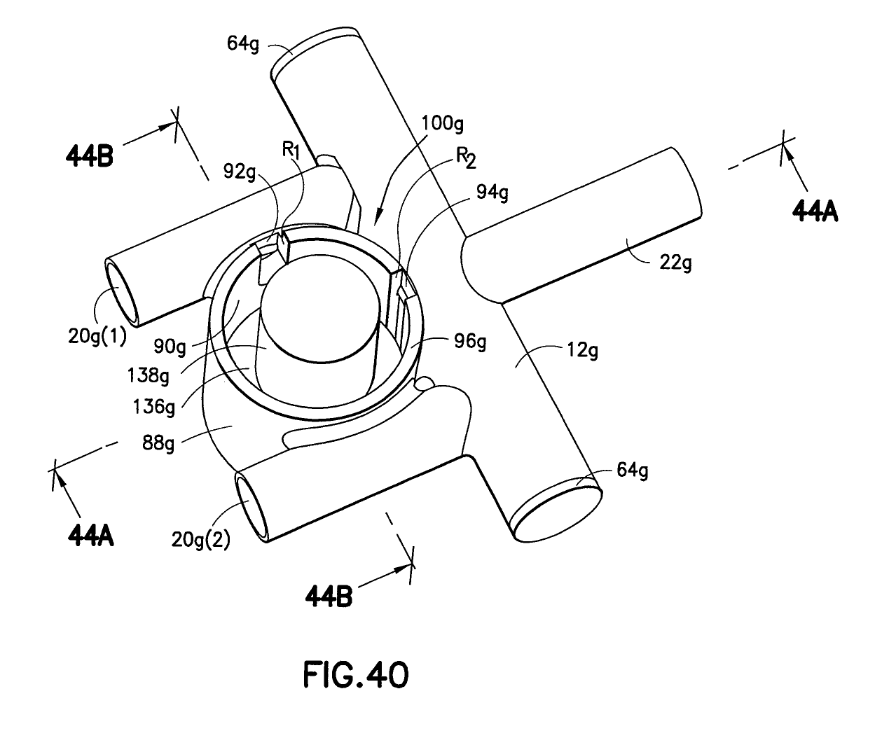

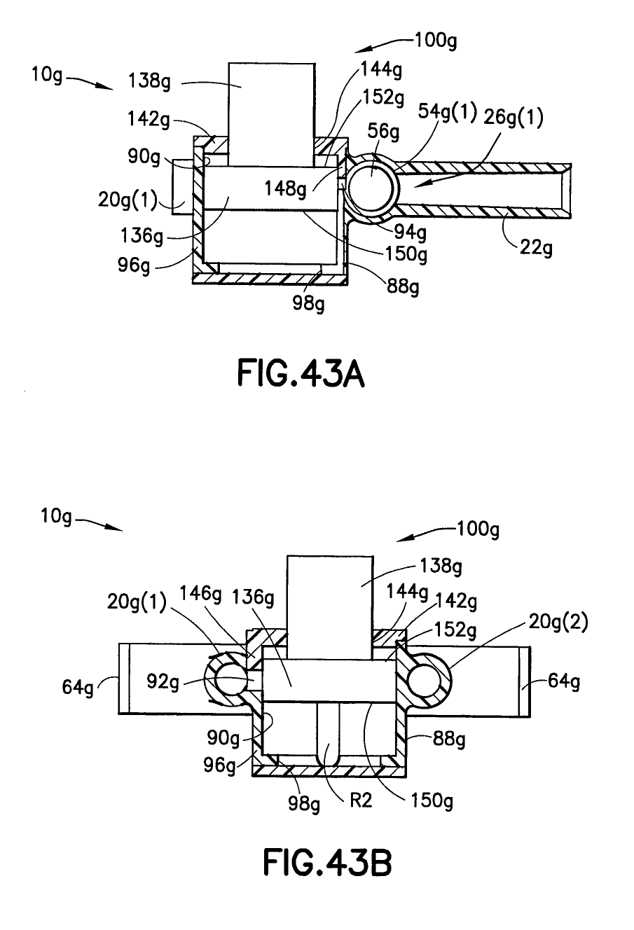

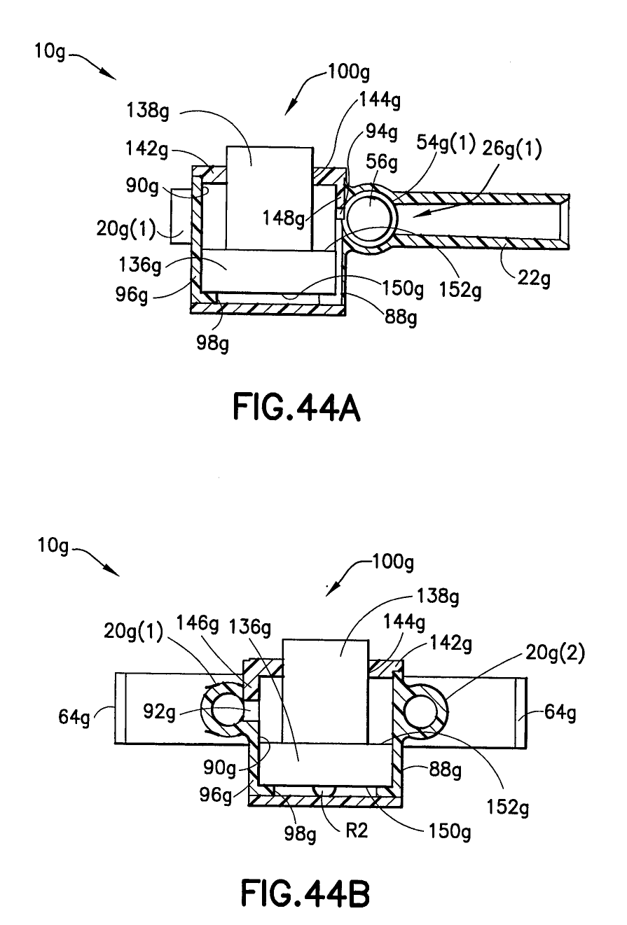

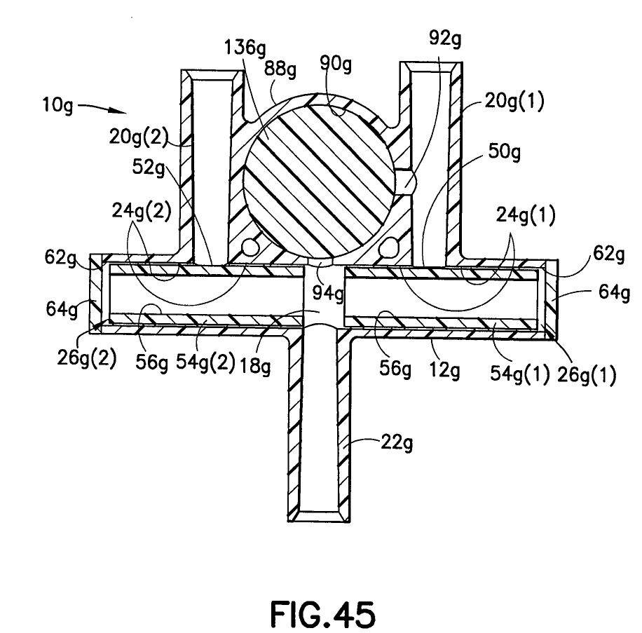

A seventh embodiment of check valve 10g is shown in FIGS. 36-45. Check valve 10g according to this embodiment comprises a housing body 12g which is substantially identical to the housing body 12f and dual valve members 26g(1), 26g(2) which are substantially identical to valve members 26f(1), 26f(2) of check valve 10f and, thus, the details of housing body 10g and valve members 26g(1), 26g(2) are not recited hereinafter. In this embodiment, cylindrical housing portion 88g and the cylindrical cavity 90g formed therein is modified slightly to accommodate and interface with override or bypass actuator 100g that is somewhat different in form and operation from cylinder lever actuator 100f discussed immediately above. Accordingly, the form and operation of override or bypass actuator 100g serve as the main differences in check valve 10g in comparison to check valve 10f discussed previously.

With respect to cylindrical housing portion 88g, side port 92g and interface port 94g are situated in the same general locations as in the cylindrical housing portion 88f of housing body 12f of check valve 10f. However, interior sidewall 96g of cylindrical housing portion 88g is recessed as represented by reference characters R₁, R₂ in the vicinity of side port 92g and interface port 94g for receiving respective depending structures from override or bypass actuator 100g as discussed herein. The recessed portion R₂ of interior sidewall 96g associated with interface port 94g extends the height of the interior sidewall 96g. Additionally, a raised rim or ledge 98g is formed at the bottom of the cylindrical cavity 90g defined by the cylindrical housing portion 88f which is broken at the location of the recessed portion R₂ of interior sidewall 96g associated with interface port 94g.

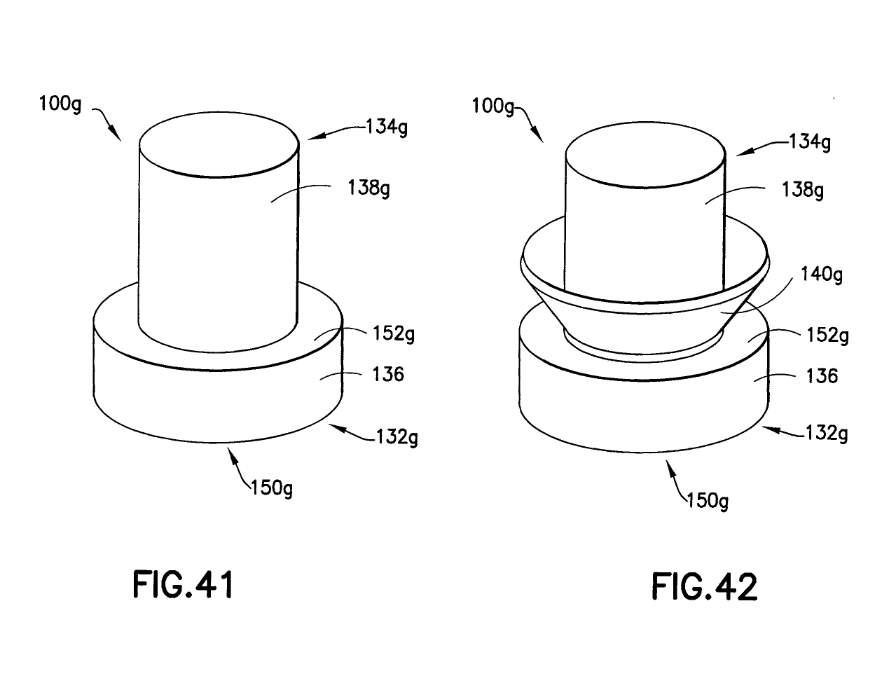

In this embodiment, override or bypass actuator 100g is again configured to override or bypass the functioning of valve member 26g(1) to permit bi-directional fluid flow between inlet port 20g(1) and outlet port 22g through the flow passage 18g but does so in a somewhat different functional manner than cylinder lever actuator 100f discussed previously. In this embodiment, override or bypass actuator 100g is in the form of a cylinder plunger actuator 100g, illustrated in isolation in FIG. 41. FIG. 42 shows an alternative variation of cylinder plunger actuator 100g. Cylinder plunger actuator 100g comprises a first or distal end 132g and a second or proximal end 134g. As depicted in FIGS. 41-42, the first or distal end 132g is formed with a plunger head 136g. A plunger stem 138g extends from plunger head 136g and defines the second or proximal end 134g which is contacted by an operator of check valve 10g to place the check valve 10g in the override or bypass position or state as discussed herein. In the alternative embodiment of cylinder plunger actuator 100g shown in FIG. 42, a sealing skirt may 140g may be provided around plunger stem 138g to improve the fluid sealing characteristics of the cylinder plunger actuator 100g when seated in cylindrical cavity 90g defined by cylindrical housing portion 88g of housing body 12g.

An annular cap member 142g is desirably provided as part of cylinder plunger actuator 100g and is seated about the plunger stem 138g. Annular cap member 142g defines a central opening 144g through which plunger stem 138g extends. Annular cap member 142g is adapted to form a sealing connection with cylindrical housing portion 88g of housing body 12g to enclose cylindrical cavity 90g defined therein and, thus, annular cap member 142g may also be considered to be a part or portion of housing body 12g. Thus, in the assembled state of cylinder plunger actuator 100g, plunger head 136g is seated within cylindrical cavity 90g and captured therein by the presence of annular cap member 142g which is desirably secured to cylindrical housing portion 88g by any of the conventional joining techniques identified previously. Plunger stem 138g passes through the central opening 144g in annular cap member 142g to be accessible to an operator of check valve 10g. Annular cap member 142g comprises two depending tab members 146g, 148g that are positioned to register with recessed portions R₁, R₂ defined in the interior sidewall 96g of cylindrical housing portion 88g. The distal ends of each of the tab members 146g, 148g are arcuate or curved in shape to complete the formation or definition of side port 92g and interface port 94g, respectively, when the tab members 146g, 148g register with recessed portions R₁, R₂. An optional and removable domed protective cap D may be provided to register or cooperate with plunger stem 138 to prevent inadvertent actuation of cylinder plunger actuator 100g by an operator.

As the normally closed position of valve members 26g(1), 26g(2) and their normal operation is substantially identical to that discussed previously in connection with check valve 10f discussed previously, a discussion of the normal operation of valve members 26g(1), 26g(2) is omitted herein. Accordingly, only the override or bypass operation of cylinder plunger actuator 100g to override or bypass the functioning of valve member 26g(1) is discussed hereinafter. In the normal operation of check valve 10g, cylinder plunger member 100g is in a normally raised first position with plunger head 136g positioned in cylindrical cavity 90g to block or seal off both side port 92g and interface port 94g. This raised or first position of cylinder plunger member 100g prevents fluid flow between side port 92g and interface 94g allowing valve members 26g(1), 26g(2) to operate as discussed previously. When it is desired to override the function of valve member 26g(1) for a patency check as an example, the operator pushes down on plunger stem 138g which has the effect of pushing plunger head 136g downward in cylindrical cavity 90g thereby exposing and opening side port 92g and interface port 94g and placing the cylinder plunger member 100g in the second or bypass position. Fluid flow may now pass directly between side port 92g and interface port 94g in cylindrical cavity 90g and vice versa for patency check purposes. Fluid flow may pass from side port 92g to interface port 94g via cylindrical cavity 90g and then onto outlet port 22g via flow passage 18g and reverse patency check fluid flow may follow the reverse path. In this embodiment, the bypass passage is defined by the flow path from side port 92g to interface port 94g via cylindrical cavity 90g which occurs when cylinder plunger member 100g is placed in the depressed, second or bypass position in cylindrical cavity 90g. Due to the configuration of cylinder plunger actuator 100g, if reverse pressurized fluid flow is encountered in flow passage 18g as, for example, if reverse pressurized fluid flow occurs in outlet port 22g, the cylinder plunger actuator 100g will automatically reset to the initial or raised position. This automatic reset feature occurs due to the greater surface area present on a bottom or under side 150g of plunger head 136g than on a top or upper side 152g of plunger head 136g that is exposed to fluid flow due to the presence of plunger stem 138g which generates a fluid pressure differential that causes the plunger head 136g to move upward in cylindrical cavity 90g. Reverse fluid flow in the foregoing situation will reach both the bottom side 150g and the upper side 152g of plunger head 136g due to the extended length of recess portion R₂ defined in the interior sidewall 96g of cylindrical housing portion 88g. Raised rim or ledge 98g allows the reverse pressurized fluid flow to act on the increased surface area bottom side 150g of plunger head 136g. Thus, cylinder plunger actuator 100g automatically resets when reverse pressurized fluid flow is present in interface port 94g.

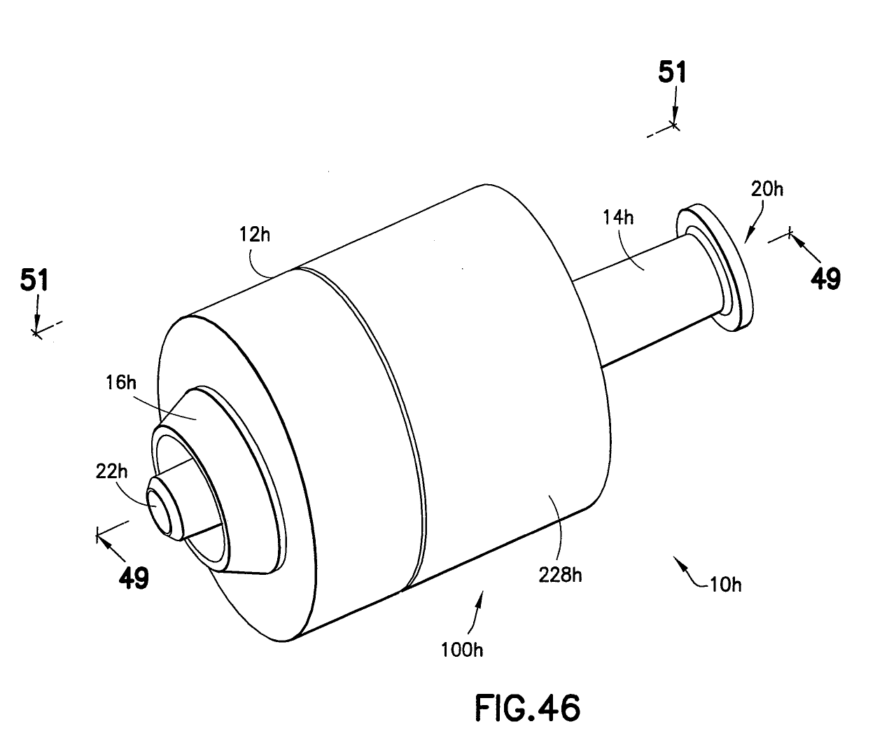

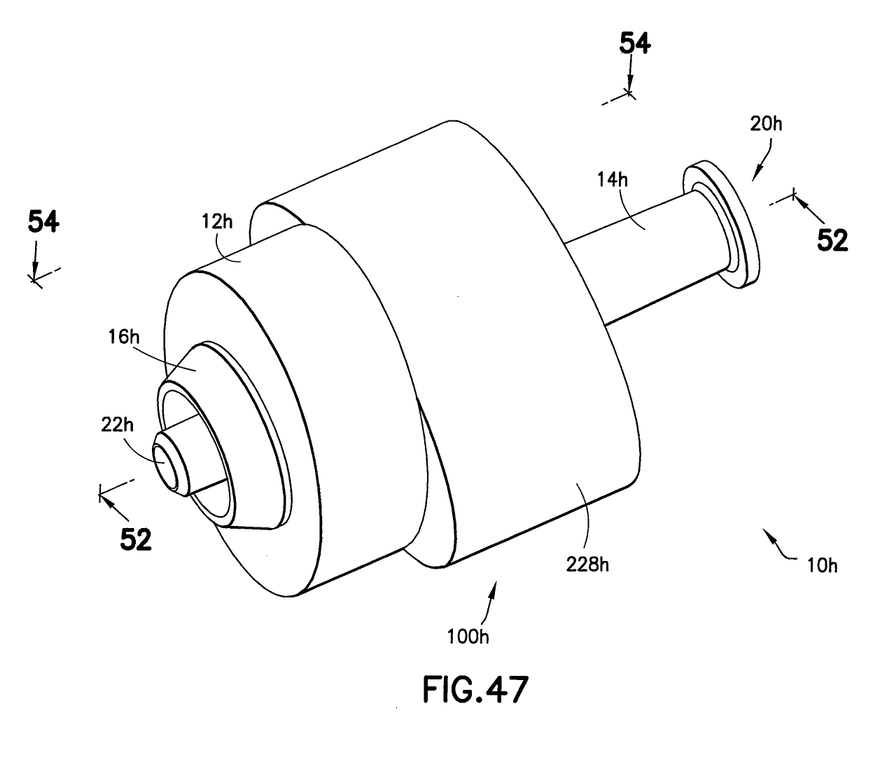

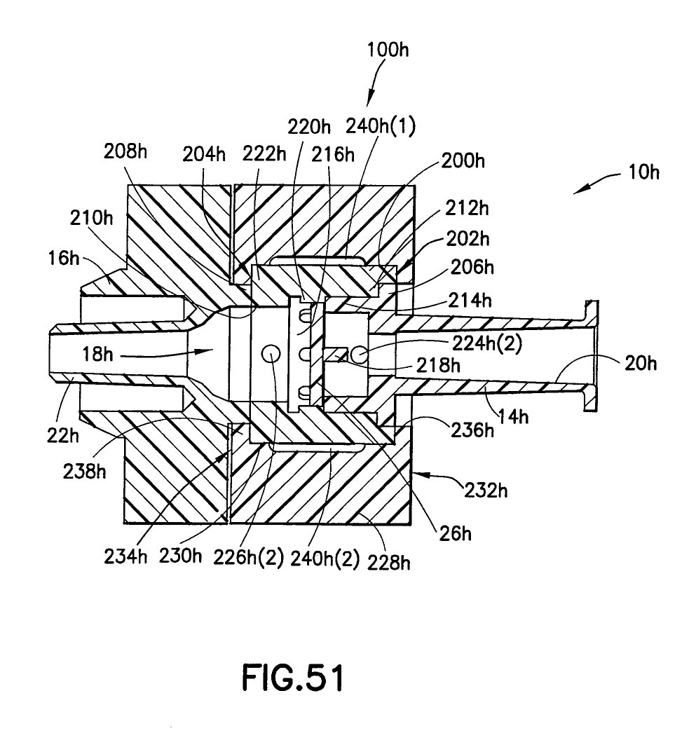

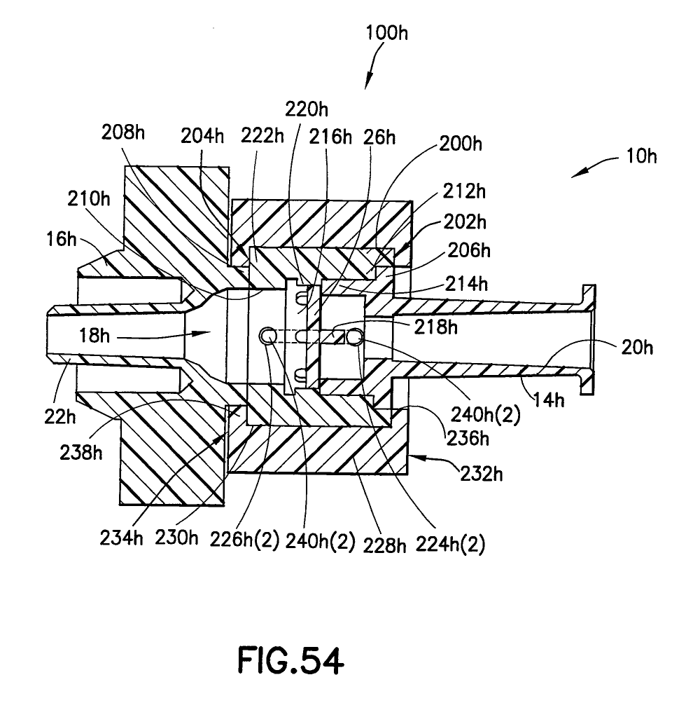

An eighth and final embodiment of check valve 10h is shown in FIGS. 46-54. Check valve 10h differs from previous embodiments in that override or bypass actuator 100h is disposed about a portion of housing body 12h and is rotationally associated therewith to place the check valve 10h in the override or bypass position or state. As a result, housing body 12h differs somewhat in form from previous embodiments and typically comprises a multi-component construction comprising, at one end, first housing portion 14h which forms or defines inlet port 20h and, at the opposing end, second housing portion 16h which forms or defines outlet port 22h. As in previous embodiments, inlet port 20h and outlet port 22h may be formed with standard luer connection configurations. A valve carrier member 200h connects the first housing portion 14h and second housing portion 16h and may be considered part of housing body 12h. Flow passage 18h is defined within valve carrier member 200h and provides fluid communication between inlet port 20h and outlet port 22h. While valve carrier member 200h is illustrated as a separate component from first housing portion 14h and second housing portion 16h it will be clear that these three individual components may be integrally formed as a singular or unitary body if so desired. The separation of these elements into three parts or components facilitates manufacture and assembly of check valve 10h and their illustration as separate components is for exemplary purposes only.

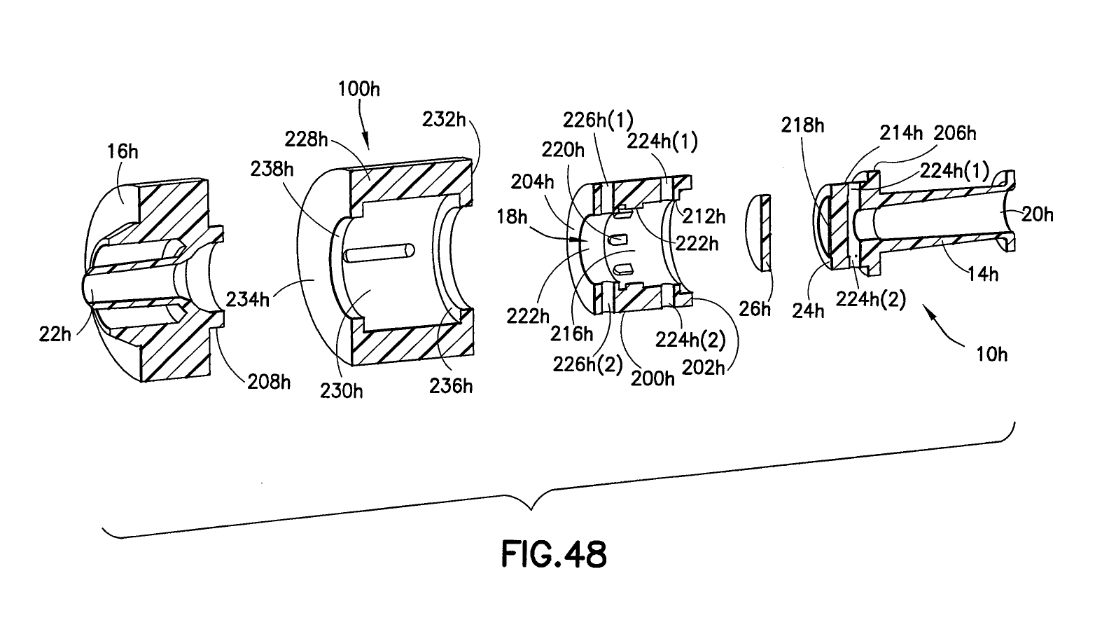

Valve carrier member 200h has a first end 202h and an opposing second end 204h. First end 202h is generally adapted to interface or join with a distal projection or flange 206h extending from first housing portion 14h and the second end 204h is generally adapted to interface or join with a proximal projection or flange 208h extending from second housing portion 16h. Valve carrier member 200h further defines a central bore 210h extending therethrough between ends 202h, 204h. Central bore 210h is stepped inward toward a central axis CL of central bore 210h at location or portion 212h to accommodate the distal projection or flange 206h of first housing portion 14h, whereby a distal portion 214h of first housing portion 14h is inserted into central bore 210h. Distal portion 214h of first housing portion 14h may be secured within the central bore 210h and distal flange 206h of the first housing portion 14h may secured in association with upstream stepped portion 212h of valve carrier member 200h by any of the conventional joining techniques identified previously. Central bore 210h defines a valve cavity 216h just distal or forward of the inserted location of distal portion 214h of first housing portion 14h in central bore 210h. Valve member 26h, which may be a conventional elastomeric disk check valve, is disposed in valve cavity 216h and adapted to interface with a seal seat 24h defined by the distal end of distal portion 214h of first housing portion 14h. As in previous embodiments, seal seat 24h is generally circular shaped as illustrated in FIG. 48. As further shown in FIG. 48, distal portion 214h of first housing portion 14h may comprise a cross member 218h to reinforce seal seat 24h and prevent disk valve member 26h from collapsing or deforming into inlet port 20h in a reverse fluid flow situation in flow passage 18h.

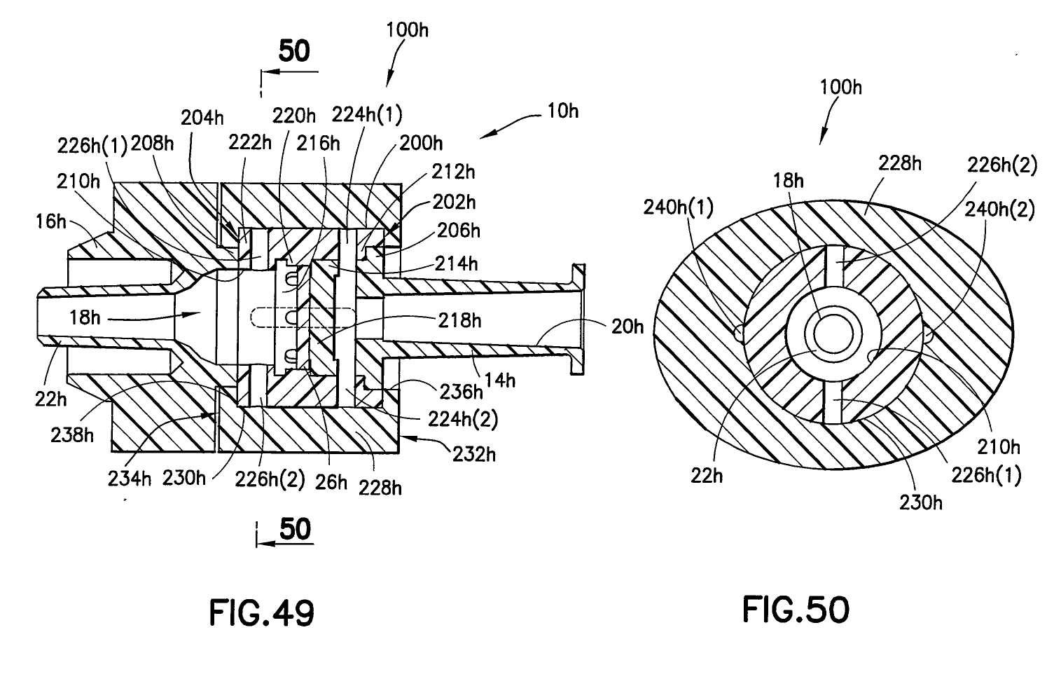

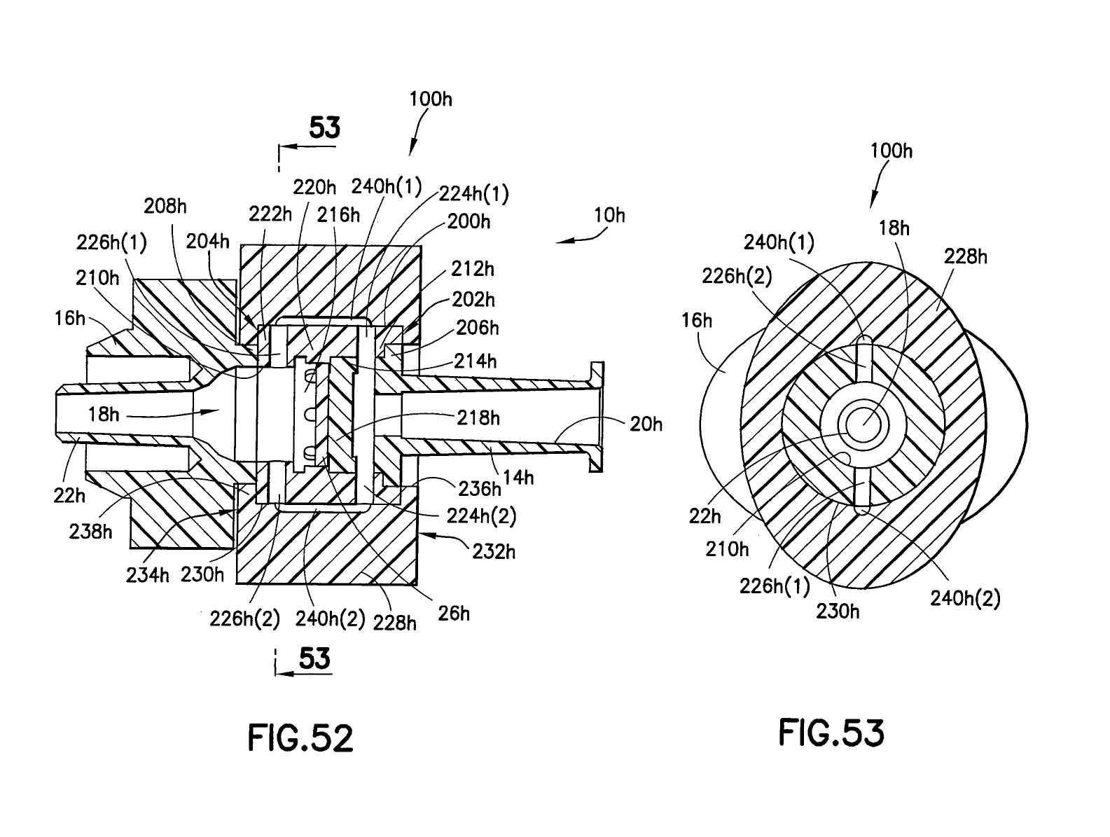

Valve carrier member 200h further comprises a plurality of inward extending tab members 220h that extend inward from the inner wall or surface of the valve carrier member 200h into valve cavity 216h. Tab members 220h extend inward from the inner periphery or surface of valve carrier member 200h into valve cavity 216h and are arranged in opposing pairs relationship with sufficient spacing therebetween to permit fluid flow from inlet port 20h to outlet port 22h in a normal or typical fluid flow situation wherein fluid flow from inlet port 20h to outlet port 22h via flow passage 18h and unseats valve member 26h from engagement with seal seat 24h defined at the distal end of the distal portion 214h of first housing portion 14h of housing body 12h. Accordingly, inward extending tab members 220h enable proper operation of check valve 10h in the normal fluid flow situation. However, sufficient frictional engagement is present between tab members 220h and the outer periphery of disk valve member 26h to prevent disk valve member 26h from moving axially downstream in flow passage 18h in the normal fluid flow situation to a position engaging a downstream stepped portion 222h of valve carrier member 200h which could cause a blockage to fluid flow in the normal fluid flow situation. Tab members 220h in part define valve cavity 216h. As shown in FIGS. 49 and 52 as examples, proximal projection or flange 208h extending from second housing portion 26h is in abutting relationship with stepped portion 222h defined by valve carrier member 200h and may be secured therewith via any of the conventional joining techniques identified previously.

Additionally, valve carrier member 200h defines two opposing pairs of ports 224h, 226h in opposing sides of valve carrier member 200h that extend through the body of the valve carrier member 200h. Ports 224h, 226h comprise a pair of first interface ports 224h(1), 224h(2) and a pair of second interface ports 226h(1), 226h(2). First interface ports 224h(1), 224(2) are formed or defined in valve carrier member 200h just distal or forward of the upstream inner stepped portion 212h of valve carrier member 200h and second interface ports 226h(1), 226h(2) are formed or defined in the downstream inner stepped portion 222h of valve carrier member 200h. The function and operation of interface ports 224h, 226h is discussed herein.

As indicated previously, override or bypass actuator 100h is disposed about housing body 12h and, in particular, about valve carrier member 200h. Accordingly, override or bypass actuator 100h is rotationally associated with valve carrier member 200h. Override or bypass actuator 100h comprises an annular actuator body 228h which defines a recessed central cavity 230h. Valve carrier member 200h is disposed in recessed cavity 230h and actuator body 228h is rotationally disposed about valve carrier member 200h. To facilitate rotational movement of actuator body 228h relative to valve carrier member 200h, valve carrier member 200h has an outer diameter slightly less than the inner diameter of recessed cavity 230h so that there is free rotational movement of actuator body 228h with respect to valve carrier member 200h. As will be apparent from FIGS. 49-54, opposing ends 232h, 234h of actuator body 228h define flanges or lips 236h, 238h of reduced internal diameter to form or define recessed cavity 230h and, further, to constrain valve carrier member 200h axially within recessed cavity 230h. Actuator body 228h further defines a pair of opposing bypass conduits 240h(1), 240h(2) in recessed cavity 230h which extend longitudinally within recessed cavity 230h and each have respective lengths equal to the distance between the first and second pairs of interface ports 224h(1), 226h(1) and 224h(2), 224h(2). Bypass conduits 240h(1), 240h(2) in recessed cavity 230h are adapted to provide fluid communication between the first and second pairs of interface ports 224h(1), 226h(1) and 224h(2), 224h(2) when actuator body 228h is rotated into a position aligning bypass conduits 240h(1), 240h(2) with the first and second pairs of interface ports 224h(1), 226h(1) and 224h(2), 226h(2).

In normal operation of check valve 10h, valve member 26h is typically initially seated against seal seat 24h defined by the distal portion 214h of first housing portion 14h as described previously. When fluid flow is present in inlet port 20h, this fluid flow acts on the “upstream” side of valve member 26h in flow passage 18h and unseats valve member 26h from seal seat 24h. As this occurs, fluid flow may pass via spacing S between the opposing sets of tab members 220h in the central bore 210h of valve carrier member 200h to allow fluid flow through flow passage 18h which, in this embodiment, is defined by the central bore 210h in valve carrier member 200h. Valve member 26h is limited in its axial downstream movement in valve cavity 216h of central bore 210h by frictional engagement with tab members 220h and fluid present on the “downstream” side of valve member 26h occurring during normal operation of check valve 10h. If reverse fluid occurs in outlet port 22h or in central bore 210h, valve member 26h is subjected to reverse fluid pressure that urges the valve member 26h into re-engagement with seal seat 24h thereby preventing reverse fluid flow into inlet port 22h. During this normal operation sequence of check valve 10h, bypass conduits 240h(1), 240h(2) not aligned with first and second pairs of interface ports 224h(1), 226h(1) and 224h(2), 226h(2) and are rotationally offset from the first and second pairs of interface ports 224h(1), 226h(1) and 224h(2), 226h(2) by approximately 90° as will be apparent when comparing FIGS. 49-51, which illustrate the normal operational state of check valve 10h, and FIGS. 52-54 which illustrate the override or bypass state of check valve 10h.

As just indicated FIGS. 49-51 illustrate the normal operational state of check valve 10h wherein bypass conduits 240h(1), 240h(2) are rotationally offset from the first and second pairs of interface ports 224h(1), 226h(1) and 224h(2), 226h(2) by approximately 90° and therefore not aligned with these ports, and FIGS. 52-54 illustrate the override or bypass state of check valve 10h wherein bypass conduits 240h(1), 240h(2) are aligned with the first and second pairs of interface ports 224h(1), 226h(1) and 224h(2), 226h(2). Check valve 10h is placed in the override or bypass state from the normal operational state by rotating actuator body 228h approximately 90° relative to valve carrier member 200h. When this rotational movement occurs, bypass conduits 240h(1), 240h(2) are aligned with the first and second pairs of interface ports 224h(1), 226h(1) and 224h(2), 226h(2), thereby forming two continuous bypass passages around valve member 26h which permit bi-directional fluid flow between inlet port 20h and outlet port 22h. In the normal operational state of check valve 10h, the inner sidewall or surface of actuator body 228h blocks fluid flow through the first and second pairs of interface ports 224h(1), 226h(1) and 224h(2), 226h(2). When actuator body 228h is rotated as described previously, bypass conduits 240h(1), 240h(2) align with the first and second pairs of interface ports 224h(1), 226h(1) and 224h(2), 226h(2) and establish fluid communication between the first and second pairs of interface ports 224h(1), 226h(1) and 224h(2), 226h(2). With the establishment of this fluid communication, bidirectional fluid flow may occur through both formed or completed bypass passages which permits check valve 10h to used for patency check applications. While check valve 10h was described with two pairs of interface ports 224h(1), 226h(1) and 224h(2), 226h(2) and two bypass conduits 240h(1), 240h(2), it will be appreciated that one pair of interface ports 224h(1), 226h(1) and a single bypass conduit 240h(1) are needed to establish the override or bypass state of check valve 10h in accordance with the foregoing.

While several embodiments of a patency check compatible check valve flow were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

What is claimed is:

1. A patency check compatible check valve, comprising:
a housing body defining a flow passage, an inlet port communicating with the flow passage, and an outlet port communicating with the flow passage, the housing body further comprising a seal seat in the flow passage between the inlet port and outlet port;
a valve member disposed in the flow passage and adapted to engage the seal seat, the valve member comprising a closed position wherein the valve member engages the seal seat and prevents fluid communication between the inlet port and outlet port and an open position permitting fluid flow from the inlet port to the outlet port in response to fluid flow in the inlet port; and
an actuator operatively connected to the valve member and adapted to place the valve member in an override position permitting bidirectional fluid flow through the flow passage.

2. A patency check compatible check as claimed in claim 1 wherein the valve member is fluid flow responsive to reverse fluid flow in the outlet port to engage the seal seat and attain the closed position

3. A patency check compatible check as claimed in claim 1 wherein the actuator comprises a lever coupled to the valve member and adapted to move the valve member to the override position.

4. A patency check compatible check valve as claimed in claim 3 wherein the lever comprises an eccentric cam coupled with the valve member such that actuation of the lever causes the eccentric cam to move the valve member to the override position.

5. A patency check compatible check valve as claimed in claim 1 wherein the valve member comprises a plunger with a seal portion adapted engage the seal seat and wherein the lever comprises an eccentric cam such that actuation of the lever causes the eccentric cam to move the valve member to the override position.

6. A patency check compatible check valve as claimed in claim 1 wherein the valve member comprises a disk member biased into engagement with the seal seat, and the actuator comprises a hand-actuated plunger coupled to the disk member such that actuation of the plunger overcomes the biasing force applied to the disk member to place the disk member in the override position.

7. A patency check compatible check valve as claimed in claim 6 wherein the disk member is biased into engagement with the seal seat by a biasing member.

8. A patency check compatible check valve as claimed in claim 7 wherein the biasing member comprises a spring.

9. A patency check compatible check valve as claimed in claim 1 wherein the valve member comprises a hollow member defining an internal flow passage in fluid communication with the flow passage of the housing body and at least one side port.

10. A patency check compatible check valve as claimed in claim 9 wherein fluid flow in the inlet port causes deformation of the hollow member to permit fluid communication between the inlet port and outlet port and place the hollow member in the open position.

11. A patency check compatible check valve as claimed in claim 10 wherein the deformation occurs along a longitudinal axis of the hollow member.

12. A patency check compatible check valve as claimed in claim 10 wherein the hollow member is resiliently deformable such that upon ceasing of fluid flow in the inlet port the hollow member resiliently returns to the closed position.

13. A patency check compatible check valve as claimed in claim 9 wherein the seal seat comprises an internal portion of the housing body.

14. A patency check compatible check valve as claimed in claim 9 wherein the actuator is coupled to the hollow member to move the hollow member axially in the flow passage of the housing body to the override position wherein the at least one side port is in fluid communication with the inlet port.

15. A patency check compatible check valve as claimed in claim 14 wherein the actuator comprises a plunger associated with an end of the hollow member such that actuation of the plunger imparts axial movement to the hollow member.

16. A patency check compatible check valve as claimed in claim 9 wherein the housing body comprises a plurality of inlet ports and the hollow member is associated with each inlet port to form the closed position therewith.

17. A patency check compatible check valve as claimed in claim 9 wherein the hollow member is tubular shaped.

18. A patency check compatible check valve, comprising:
a housing body defining a flow passage, an inlet port communicating with the flow passage, and an outlet port communicating with the flow passage, the housing body further comprising a seal seat in the flow passage between the inlet port and outlet port;
a cantilever valve member disposed in the flow passage and adapted to engage the seal seat, the cantilever valve member comprising a closed position wherein the cantilever valve member engages the seal seat and prevents fluid communication between the inlet port and outlet port and an open position permitting fluid flow from the inlet port to the outlet port in response to fluid flow in the inlet port.

19. A patency check compatible check valve as claimed in claim 18 wherein the seal seat comprises an internal portion of the housing body.

20. A patency check compatible check valve as claimed in claim 18 wherein the cantilever valve member comprises a resilient leaf spring.

21. A patency check compatible check valve as claimed in claim 18 wherein the housing body comprises two inlet ports and the cantilever valve member is fluid flow responsive such that fluid flow in one of the two inlet ports causes the cantilever valve member to form the closed position with the other inlet port.

22. A patency check compatible check valve, comprising:
a housing body defining a flow passage, a first inlet port communicating with the flow passage, a second inlet port communicating with the flow passage, and an outlet port communicating with the flow passage, the first and second inlet ports each comprising an inlet port member extending into the flow passage from opposing sides;
a valve member disposed in the flow passage and comprising opposing recesses receiving the opposing first and second inlet port members and adapted to form a fluid seal with the opposing first and second inlet port members, the valve member being fluid flow responsive to fluid flow in one or both of the first and second inlet ports to form multiple states comprising:
a first state wherein fluid communication between the first inlet port and the outlet port is present while a fluid seal is present between the second inlet port and the outlet port;
a second state wherein fluid communication between the second inlet port and the outlet port is present while a fluid seal is present between the first inlet port and the outlet port; and
a third state wherein fluid communication is at least partially present between both the first inlet port and the second inlet port and the outlet port.

23. A patency check compatible check valve as claimed in claim 22 wherein the valve member is cylindrical shaped and the opposing recesses are defined in opposite ends of the cylindrical valve member.

24. A patency check compatible check valve as claimed in claim 22 wherein the first and second inlet port members are segmented.

25. A patency check compatible check valve as claimed in claim 22 wherein the first and second inlet port members are each formed as a slotted dome.

26. A patency check compatible check valve, comprising:
a housing body defining a flow passage, an inlet port communicating with the flow passage, and an outlet port communicating with the flow passage, the housing body further comprising a seal seat in the flow passage between the inlet port and outlet port;
a valve member disposed in the flow passage and adapted to engage the seal seat, the valve member comprising a closed position wherein the valve member engages the seal seat and prevents fluid communication between the inlet port and outlet port and an open position permitting fluid flow from the inlet port to the outlet port in response to fluid flow in the inlet port; and
a bypass actuator defining at least in part a bypass passage and adapted to selectively place the inlet port in fluid communication with the outlet port, the bypass actuator having a first position wherein fluid flow through the bypass passage to the outlet port is prevented and a bypass position wherein fluid communication is enabled between the inlet port and the outlet port via the bypass passage.

27. A patency check compatible check valve as claimed in claim 26 wherein valve member comprises a hollow member defining an internal flow passage in fluid communication with the flow passage of the housing body.

28. A patency check compatible check valve as claimed in claim 27 wherein fluid flow in the inlet port causes deformation of the hollow member to permit fluid communication between the inlet port and outlet port and place the hollow member in the open position.

29. A patency check compatible check valve as claimed in claim 28 wherein the deformation occurs along a longitudinal axis of the hollow member.

30. A patency check compatible check valve as claimed in claim 28 wherein the hollow member is resiliently deformable such that upon ceasing of fluid flow in the inlet port the hollow member resiliently returns to the closed position.

31. A patency check compatible check valve as claimed in claim 27 wherein the hollow member is tubular shaped.

32. A patency check compatible check valve as claimed in claim 26 wherein the seal seat comprises an internal portion of the housing body.

33. A patency check compatible check valve as claimed in claim 26 wherein the housing body comprises a plurality of inlet ports and a valve member is associated with each inlet port to form the closed position therewith.

34. A patency check compatible check valve as claimed in claim 26 wherein the valve member comprises a disk member adapted to seat against the seal seat.

35. A patency check compatible check valve as claimed in claim 26 wherein the bypass actuator is adapted for rotational movement to select between the first position and the bypass position.

36. A patency check compatible check valve as claimed in claim 26 wherein the bypass actuator comprises a plurality of bypass passages to enable fluid communication between the inlet port and the outlet port via multiple bypass passages.

37. A patency check compatible check valve as claimed in claim 36 wherein the bypass actuator is adapted for rotational movement to select between the first position and the bypass position.

38. A patency check compatible check valve as claimed in claim 26 wherein the bypass actuator comprises a bypass plunger disposed in a cavity defined by the housing body, and wherein in the first position the bypass plunger prevents fluid flow through the bypass passage and in the bypass position at least in part defines the bypass passage such that fluid communication is enabled between the inlet port and the outlet port.

39. A patency check compatible check valve as claimed in claim 38 wherein the first position comprises a raised position of the bypass plunger in the cavity and the bypass position comprises a depressed position of the bypass plunger in the cavity.

40. A patency check compatible check valve as claimed in claim 38 wherein the bypass plunger comprises a plunger head seated in the cavity and a plunger stem extending outward from the housing body, and wherein a bottom side of the plunger head defines a greater fluid contacting surface area than a top side of the plunger head such that reverse fluid flow in the outlet port automatically returns the bypass plunger to the first position.
